(19) United States
(12) Patent Application Publication
(10) Pub. No.: US 2026/0076898 A1
(43) Pub. Date: Mar. 19, 2026

(54) Method For Treating X-Linked Retinoschisis (XLRS) And/Or For Reducing Intraretinal Cysts In A Subject

(71) Applicant: UNIVERSITY OF IOWA RESEARCH FOUNDATION, Iowa City, IA (US)

(72) Inventors: Arlene V. Drack, Iowa City, IA (US); Jacob M. Thompson, Iowa City, IA (US); Ella J. Gehrke, Iowa City, IA (US); Salma H. A. Hassan, Iowa City, IA (US); Ying Hsu, Iowa City, IA (US)

(21) Appl. No.: 19/319,477

(22) Filed: Sep. 4, 2025

Related U.S. Application Data
(60) Provisional application No. 63/696,394, filed on Sep. 19, 2024.

(51) Int. Cl. A61K 9/00; A61K 9/08; A61K 31/433; A61K 31/542; A61K 47/02; A61K 47/10; A61P 27/02

(52) U.S. Cl. CPC A61K 9/0048; A61K 9/08; A61K 31/433; A61K 31/542; A61K 47/02; A61K 47/10; A61P 27/02

(57) ABSTRACT

Disclosed are methods for treating X-linked retinoschisis (XLRS) in a subject in need thereof, for reducing intraretinal cysts, and/or preventing the formation of intraretinal cysts in a subject in need thereof.

### CROSS-REFERENCE TO RELATED APPLICATIONS

This application claims the benefit of U.S. Provisional Application Ser. No. 63/690,394, filed Sep. 4, 2024, the entire content of which is incorporated by reference in its entirety.

### SEQUENCE LISTING STATEMENT

An electronic sequence listing (13976600127.xml; Size: 3,622 bytes; and Date of Creation: Sep. 4, 2025) accompanies this application. The sequence listing is incorporated by reference in its entirety.

### BACKGROUND

X-linked retinoschisis (XLRS) is a vitreoretinal dystrophy caused by RS1 gene mutations, which disrupt retinoschisin-1 (RS1) function. Vital for retinal architecture, the absence of functional RS1 leads to the development of intraretinal cysts. Intravitreal injection of a gene therapy for treating XLRS caused ocular inflammation in high dose groups in a phase I/II clinical trial. New therapies and methods are needed for preventing or treating retinoschisis or XLRS.

### SUMMARY

Disclosed are methods for preventing or treating X-linked retinoschisis (XLRS) in a subject in need thereof, for reducing intraretinal cysts and for preventing the formation of intraretinal cysts in a subject.

One aspect is a method of preventing or treating XLRS in a subject in need thereof, the method comprising: administering an aqueous composition to an intravitreal space of an eye of a subject in need thereof.

A second aspect is a method of reducing intraretinal cysts and/or preventing the formation of intraretinal cysts in a subject in need thereof, the method comprising administering an aqueous composition to an intravitreal space of an eye of a subject in need thereof.

Another aspect is a method of treating XLRS and/or reducing intraretinal cysts in a subject in need thereof, the method comprising: administering topical and/or subconjunctival hypertonic solutions; rapid lowering of intraocular pressure in the eye of the subject using medical and/or surgical techniques; and administering an aqueous composition to an intravitreal space of the eye of the subject.

In all disclosed aspects, the aqueous composition comprises a buffered saline solution that may be a hypertonic buffered saline solution or an isotonic buffered saline solution.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The drawings described below are for illustration purposes only and are not intended to limit the scope of the disclosed subject matter in any way.

FIGS. 1A-1D show natural history of disease in the Rs1-KO mouse compared to WT BL/6 mouse. OCT images were collected from p15 to 9-months of age in Rs1-KO mice. Representative OCT images demonstrate retinoschisis in Rs1-KO mice at p15, 1-, 2- and 4-months old, compared to a WT mouse without retinoschisis (FIG. 1A). Cyst severity (FIG. 1B) and ONL thickness (FIG. 1C) were measured at each age. For cyst severity, cyst height was measured at 4 different positions in an eye and translated to a scoring scale of 1 to 6, with 1 representing a cyst height of 0. Electroretinographies (ERGs) of Rs1-KO mice and WT mice were collected at 2- and 6-months of age. As early as 2-months of age, Rs1-KO mice show reduced ERG b-wave amplitudes and delayed latency of the b-wave peak, compared to the robust WT waveforms (FIG. 1D). Layers of the WT mouse retina is represented as follows; retinal nerve fiber layer and ganglion cell layer (RNFL+GC), inner plexiform layer (IPL), inner nuclear layer (INL), outer plexiform layer (OPL), outer nuclear layer (ONL), external limiting membrane (ELM), photoreceptor inner and outer segments (IS/OS), retinal pigment epithelium (RPE), and choroid (AL).

FIGS. 2A-2C show cyst severity is significantly reduced in vector-treated and hypertonic buffer-injected eyes at 1 month post injection (MPI). OCT images of vector-treated, hypertonic buffer-injected, and untreated eyes were collected at 1, 2, and 5 MPI. Volumetric scans were taken centering on the optic nerve. Cyst severity (FIG. 2A) was determined via manual segmentation of cyst area using the Image J software. Multiple comparisons were performed between every group at each time point. Non-significant comparisons were not annotated in the graphs. Vector-treated (n=10) or hypertonic buffer-injected Rs1-KO eyes demonstrated significantly less cyst severity than untreated eyes (FIG. 2B) at 1 MPI. ONL thickness (FIG. 2C) remained stable from 1 to 5 MPI in all cohorts; however, at 5 MPI, vector treated eyes had statistically less cyst severity than untreated eyes.

FIGS. 3A-3E show hypertonic buffer robustly outperforms untreated and vector-treated eyes in light-adapted ERGs. Cone-dependent retina function was measured using two separate light-adapted ERG assays—the 5 Hz Flicker (FIG. 3A) and 3.0 flash (FIG. 3B). ERG amplitudes are shown from 1-month post injection (MPI) to 5 MPI for vector-treated, hypertonic buffer-injected, and untreated eyes in Rs1-KO mice, and representative ERG traces at 5 MPI are illustrated. In response to both light-adapted assays, hypertonic buffer-injected eyes showed higher amplitudes than untreated and vector-treated eyes to 5 MPI (FIG. 3A). The combined rod-cone function was measured by the 3.0 cd·s/m² standard combined response (SCR) under dark-adapted conditions (FIG. 3C). At 1, 3, and 5-MPI, there was no difference between vector-treated, hypertonic buffer-injected, or untreated eyes. Isolated rod function was measured using 0.01 cd·s/m² dim flash after dark adaptation (FIG. 3D). There was no persistent significant difference between hypertonic buffer-injected, isotonic buffer-injected, or untreated eyes in rod dominant ERG settings.

FIGS. 4A-4B show vector-treated and hypertonic buffer-injected Rs1-KO mice perform faster in a light-adapted swim maze than untreated Rs1-KO mice. Mice were placed in a swim maze and testing was performed under light-adapted (FIG. 4A) or dark-adapted (FIG. 4B) conditions. Time-to-platform was recorded. Each data point is representative of the average time-to-platform for a single mouse. In light-adapted conditions, we observed that vector-treated mice (2.80 seconds) and hypertonic buffer-injected mice (3.05 seconds) had a generally faster trend in average swim performance when finding a random platform than untreated mice (4.04 seconds) in lighted environments. In a dark-adapted environment, there is a less impactful effect on average time-to-platform between vector-treated (11.31 seconds), hypertonic buffer-injected (12.99 seconds), and untreated eyes (12.73 seconds), suggesting a limited effect on visual function in the dark (B).

FIGS. 5A-5C show cyst area is reduced in hypertonic buffer and isotonic buffer-injected eyes, with greater reduction observed in the hypertonic buffer group. OCT images were collected at 2- and 3-weeks post injection (WPI), 1-month post-injection (MPI), and 2 MPI. Cyst area quantification (FIG. 5A) and outer nuclear layer (ONL) thickness (FIG. 5B) measurements were taken to assess cyst severity and photoreceptor cell survival, respectively. Representative OCT images demonstrate cysts at respective time points in a hypertonic buffer-injected mouse (FIG. 5C). Each data point is representative of one eye. At 3 WPI, there was less cyst severity in the hypertonic buffer-injected eyes compared to untreated eyes (FIG. 5A). At 1 MPI, hypertonic and isotonic buffer-treated eyes both have less severe cysts than untreated and sham punctured eyes. This effect persisted until 2 MPI for the hypertonic buffer-injected eyes compared to untreated and sham punctured eyes. ONL thickness remains stable to 5 MPI and does not significantly differ between injection groups and untreated controls (FIG. 5B).

FIGS. 6A-6E show hypertonic buffer-injected eyes experience a robust rescue of cone functioning. Cone-dependent retina function was measured using two separate light-adapted ERG assays—the 3.0 cd·s/m² flash (FIG. 6A), and the 5 Hz Flicker (FIG. 6B) in hypertonic buffer, isotonic buffer, and sham punctured eyes in Rs1-KO mice. Each data point represents one eye. ERG amplitudes are shown from 1-month post injection (MPI) to 5 MPI. In both light-adapted assays (FIGS. 6A-6B), hypertonic and isotonic buffer-injected eyes showed higher amplitudes compared to untreated eyes at 1 MPI. Hypertonic buffer-injected eyes showed improvements over untreated and sham punctured eyes until 5 MPI. The combined rod-cone function was measured by the 3.0 cd·s/m² SCR under dark-adapted conditions (FIG. 6C). Rod-dependent retina function was measured by the 0.01 cd·s/m² dim flash (FIG. 6D). In the 0.01 dim flash, hypertonic buffer-injected eyes showed improvements over untreated at 1 MPI and over both untreated and isotonic buffer-injected eyes at 2 MPI. However, significant differences over time were not observed (FIG. 6E).

FIGS. 7A-7F show peak cyst severity on OCT is negatively associated with cone function on ERG at 5 MPI. Peak cyst severity of hypertonic buffer-injected, isotonic buffer-injected, sham-punctured and untreated eyes was determined by comparing the cyst area measurements of each eye at 1, 2, and 5 MPI. The cyst area of each eye representing their peak severity was correlated with its ERG amplitudes at 5 MPI. Each dot represents one eye. A negative correlation was found between cyst area and ERG amplitudes in the light-adapted 5 Hz Flicker and 3.0 cd·s/m² flash (FIGS. 7A, 7B). Little to no correlation was found between peak cyst area and ERG amplitudes in dark-adapted 0.01 cd·s/m² or 3.0 cd·s/m² SCR (FIGS. 7C, 7D, 7E, 7F). These findings suggest increasing cyst area has greater impact on impairing cone electrical function, than rod photoreceptors.

FIGS. 8A-8B show increased cone density at 5 months after subretinal injections of a hypertonic or an isotonic buffer. Retinal sections were collected from untreated Rs1-KO eyes, hypertonic buffer-injected eyes, isotonic buffer-injected eyes, and sham puncture eyes at approximately 6-months of age. Sections were stained by PNA to visualize cone outer segments. The number of cone outer segments per 100 μm of the retina was quantified by three participants masked to treatment groups, and quantifications by these three participants were averaged. Representative immunofluorescent staining (red: PNA) of cone outer segments (arrow), demonstrating increased density of cones in hypertonic buffer-injected and isotonic buffer-injected eyes compared to untreated eyes with sparse cone staining (FIG. 8A). Synapses were visualized by an anti-RIBEYE antibody (green). Each data point represents the averaged cone density of one eye. Hypertonic buffer-injected eyes and isotonic buffer-injected eyes show significantly higher cone density per 100 m than untreated Rs1-KO eyes (FIG. 8B).

FIGS. 9A-9C show subretinal injection of hypertonic buffer alone does not enhance ERG amplitudes in mice without XLRS. To determine if the introduction of a hypertonic buffer has a stand-alone effect on ERG amplitudes, a cohort of mice without XLRS were subretinally injected with the buffer. ERGs were collected at 2, 3, and 6 MPI. Cone function was assessed using two light-adapted ERG assays: the 5 Hz Flicker and the 3.0 cd·s/m² flash. In response to both the 3.0 cd·s/m² flash and 5 Hz flicker assays, hypertonic buffer-injected eyes showed no significant change in amplitude compared to untreated eyes. This demonstrates that the hypertonic buffer alone does not enhance ERG metrics, confirming that the observed improvements in cone-specific functioning in hypertonic buffer-injected Rs1-KO eyes are genuine. Representative waveforms at 6 MPI illustrate these findings, even indicating a pattern of mild reduction in amplitude associated with the subretinal injection of hypertonic buffer (FIG. 9C).

FIGS. 10A-10C show spatial distribution of cone counts in regions superior or inferior to the optic nerve. Spider plot shows total cone count per image for individual mice at various distances from the optic nerve head (A) and the averaged cone counts per image across treatment groups (*: untreated vs isotonic buffer: p=0.0224, untreated vs puncture: p=0.0106) (#: hypertonic vs untreated: 0.0268, untreated vs isotonic: p=0.0004) (B). Within treatment groups, there is minimal variability in cone distribution across the studied area. Representative 4× immunofluorescent image shows the placement of individual 40× images along the retina and the areas sampled (FIG. 10C).

FIGS. 11A-11G show quantification of cones normalized to untreated fellow eyes demonstrates a trend for higher cone counts in hypertonic buffer-injected eyes. Flat mounts of mouse retinas were collected from untreated eyes and from eyes treated with hypertonic, isotonic, and sham punctured eyes. Sections were stained with PNA to visualize cone outer segments. The number of cones, found in two separate quadrants of an area approximately 350 μm², were quantified using ImageJ software and confirmed by masked participants, then averaged. Data were normalized to determine the percentage of cones per eye compared to the untreated fellow eye. (FIG. 11A) Percent cone count normalized to untreated eye. The results suggest a trend toward a higher percentage of cones in hypertonic buffer-injected eyes, although this increase was not statistically significant. (FIG. 11B) Representative flat mount of a retina from an eye injected with hypertonic buffer. (FIG. 11C) Close-up view of the area in one quadrant of the retina shown in (FIG. 11B), highlighting cone outer segments. (FIG. 11D) Representation of individual cone counting by ImageJ software. (E) Representative flat mount of a retina from an untreated eye. (FIG. 11F) Close-up view of the area in one quadrant of the retina shown in (FIG. 11E), highlighting cone outer segments. (FIG. 11G) Representation of individual cone counting by ImageJ software.

FIGS. 12A-12D show light-adapted ERG responses following intravitreal buffer injections. (FIG. 12A) B-wave amplitudes from the 3.0 flash LA test showing significantly higher responses in hypertonic-treated eyes vs. untreated eyes at all timepoints, and vs. isotonic-treated eyes at 2 and 3 MPI. (FIG. 12B) Representative 3.0 flash ERG waveforms at 2 WPI and 5 MPI across all groups. (FIG. 12C) 5 Hz flicker amplitudes showing significant improvement in hypertonic-treated eyes compared to untreated eyes at all timepoints and isotonic-treated eyes at 2 and 3 MPI. (FIG. 12D) Representative 5 Hz flicker waveforms at 2 WPI and 5 MPI.

FIGS. 13A-13B show cyst severity following intravitreal buffer injections. (FIG. 13A) Representative OCT images at 2 WPI from untreated, hypertonic-treated, and isotonic-treated eyes. (FIG. 13B) Quantification of cyst area (measured using Photoshop) shows a significant reduction in hypertonic-treated eyes vs. untreated eyes at 2 WPI, with a consistent trend of reduced cyst size in hypertonic-treated eyes across later timepoints compared to both isotonic treated and untreated groups.

FIGS. 14A-14B are a comparison between intravitreal and subretinal hypertonic buffer injections. (FIG. 14A) B-wave amplitudes from the 3.0 flash LA ERG and (FIG. 14B) 5 Hz flicker ERG show no significant differences between intravitreal and subretinal hypertonic buffer injections, suggesting both routes produce comparable functional rescue effects.

### DETAILED DESCRIPTION

Disclosed are methods for preventing or treating X-linked retinoschisis (XLRS) in a subject in need thereof, methods for reducing intraretinal cysts in a subject, and methods of preventing the formation of intraretinal cysts in a subject. “X-linked retinoschisis (XLRS)” as used herein refers to a vitreoretinal dystrophy caused by one or more RS1 gene mutations and/or to the development of intraretinal cysts. This disclosure presents evidence that intraretinal cyst resolution can be triggered through an osmolarity-dependent pathway. Early cyst resolution has long-term effects on cone signaling and cone survival.

### Methods

One aspect of the disclosure is a method of treating X-linked retinoschisis (XLRS) in a subject in need thereof that includes, but is not limited to, administration of or delivering an aqueous composition comprising a buffered saline solution to an intravitreal space of an eye of the subject.

In aspects, the buffered saline solution is a hypertonic buffered saline solution or an isotonic buffered saline solution.

Another aspect is a method of preventing X-linked retinoschisis (XLRS) in a subject in need thereof includes, but is not limited to, administration or delivery of an aqueous composition comprising a buffered saline solution to an intravitreal space of an eye of the subject. In aspects, the buffered saline solution is a hypertonic buffered saline solution or an isotonic buffered saline solution.

In another aspect, a method of reducing intraretinal cysts and/or preventing the formation of intraretinal cysts in a subject in need thereof is provided. The method comprises administering or delivering an aqueous composition comprising a buffered saline solution to an intravitreal space of an eye of a subject. In the aspects, the buffered saline solution is a hypertonic buffered saline solution or an isotonic buffered saline solution.

In the disclosed aspects, the methods may further comprise topically administering one or more carbonic anhydrase inhibitors to the eye of the subject. Exemplary carbonic anhydrase inhibitors include acetazolamide, dichlorphenamide/diclofenamide, methazolamide, dorzolamide, brinzolamide, and ethoxzolamide. In the disclosed aspects, the method may further comprise reducing pressure within a vitreous cavity of the eye of the subject. In the aspects, administering one or more carbonic anhydrase inhibitors and/or reducing pressure within a vitreous cavity of the eye of the subject may be before or after administering an aqueous composition comprising a buffered saline solution to an intravitreal space of the eye.

A further aspect is a method of treating XLRS and/or reducing intraretinal cysts in a subject in need thereof comprising: administering or delivering a topical hypertonic solution and/or a subconjunctival hypertonic solution the eye of the subject: using medical and/or surgical techniques for rapid lowering of intraocular pressure in the eye of the subject; and administration or delivery of an aqueous composition comprising a buffered saline solution to an intravitreal space of an eye of the subject. In aspects, the buffered saline solution is a hypertonic buffered saline solution or an isotonic buffered saline solution. Medical and surgical techniques for rapid lowering of intraocular pressure in the eye are known to those in the art, which may include prescription eye drops, oral medicines, laser treatment, as well as surgery. Such techniques decrease fluid production and/or increase fluid drainage in the eye. In all aspects, delivery of the aqueous composition may be before or after delivery of the topical hypertonic solution and/or a subconjunctival hypertonic solution.

The term “subject” may be used interchangeably with the terms “individual” and “patient” and includes human and non-human subjects. In some embodiments, the subject has one or more RS1 gene mutations and/or one or more intraretinal cysts. The subject can be any age. In certain embodiments, the subject can be less than 80 years old, less than 70 years old, less than 60 years old, less than 50 years old, less than 40 years old, less than 30 years old, less than 20 years old, less than 18 years old, less than 15 years old, less than 12 years old, or less than 10 years old; and/or at least 3 years old, at least 4 years old, or at least 5 years old.

“Intravitreal space” refers to the vitreous chamber or space of the eye, which is located behind the lens of the eye, in certain aspects. The intravitreal space is distinct from the subretinal and retinal spaces of the eye.

In aspects, the subject has one or more RS1 gene mutations. XLRS is caused by RS1 gene mutations, which disrupt retinoschisin-1 (RS1) function. The absence of functional RS1 leads to the development of intraretinal cysts. In the disclosed aspects, an aqueous composition comprising a buffered saline solution is administration to an intravitreal space of an eye of a subject having one or more RS1 gene mutations to treat XLRS, or prevent or reduce intraretinal cysts. In aspects, the buffered saline solution is a hypertonic buffered saline solution or an isotonic buffered saline solution.

In aspects, the subject is at risk of having or has one or more intraretinal cysts. In the disclosed aspects, an aqueous composition comprising a buffered saline solution may be administered to an intravitreal space of an eye of a subject at risk of having or having intraretinal cysts. One aspect is a method of preventing or reducing intraretinal cysts in a subject at risk of having or having intraretinal cysts or XLRS. In aspects, the buffered saline solution is a hypertonic buffered saline solution or an isotonic buffered saline solution.

In aspects, the aqueous composition does not include a therapeutic agent, such as a gene therapy.

In the disclosed aspects, a buffered saline solution comprises about 100 mM to about 400 mM NaCl, or about 100 mM, about 150 mM, about 200 mM, about 250 mM, about 300 mM, about 350 mM, or about 400 mM NaCL. In some aspects, the buffered saline solution comprises about 155 mM NaCl, or about 180 mM NaCl. In some aspects, the buffered saline solution is a hypertonic saline solution. In embodiments, a hypertonic saline solution comprises about 180 mM NaCl. In some aspects, the buffered saline solution is an isotonic saline solution. In embodiments, a isotonic saline solution comprises about 155 mM NaCl.

In embodiments, the buffered saline solution further comprises PBS at pH 7.0 to pH 7.4, and 0.001 wt. % of a polyoxyethylene-polyoxypropylene block copolymer, for example Plutonic F68® (F68). F68 is a non-ionic detergent commonly used in the art. In embodiments, the buffered saline solution comprises about 100 mM to about 400 mM NaCl, PBS at pH 7.0 to pH 7.4, and 0.001 wt. % of F68. In some embodiments, the buffered saline solution comprises 180 mM NaCl, 0.001% F68 in PBS, at a pH 7.4. In some embodiments, the buffered saline solution comprises 155 mM NaCl, 0.001% F68 in PBS, at a pH 7.0.

Unexpectedly, hypertonic buffer injected into eyes results in reduced cyst severity, higher amplitudes in cone-dominant electroretinography (ERG), and improved navigational vision in the light, compared to untreated Rs1-KO eyes. Surprisingly, hypertonic buffer administered to an eye, such as injected to an eye, results in greater reduction in cyst severity and improved cone-dominant ERG metrics, compared to isotonic buffer-injected eyes, and untreated Rs1-KO eyes or sham puncture eyes.

In embodiments, hypertonic buffer delivered to eyes preserves cone cells. For example, hypertonic buffer delivered to the eye results in greater cone density compared to untreated Rs1-KO eyes.

### Definitions and Terminology

The definitions and terminology used in this disclosure are for the purpose of describing particular embodiments only, and are not intended to be limiting.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, 5, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

In various aspects, the aqueous composition can include one or more components to form an isotonic or hypertonic composition. In certain aspects, the one or more components can be compounds that provide the desired osmolarity and/or tonicity. In one aspect, the one or more components can include a salt (such as, for example, NaCl, KCl, or NaHCO₃) and/or other components, such as mannitol. In certain aspects, the aqueous composition can include one or more buffering agents.

As used herein, the term “buffer” refers to a pH buffering agent that helps maintain the pH of an aqueous solution. Non-limiting examples of pH buffering agents include phosphate buffers (e.g. PBS), Tris, citric acid, histidine, glycine, HEPES, MOPS, and PIPES.

### EXAMPLES

X-linked retinoschisis (XLRS) is a vitreoretinal dystrophy caused by RS1 gene mutations which disrupt retinoschisin-1 (RS1) function. Vital for retinal architecture, the absence of functional RS1 leads to the development of intraretinal cysts. Juvenile XLRS is the leading cause of macular dystrophy in young males, with a global prevalence of up to 1 in 5,000 individuals (1). The pathology of the disease involves an X-linked recessive mutation in the retinoschisin-1 (RS1) gene located at Xp22.1-p22.3 (1). Mutations in RS1, of which there are greater than 200 known, impair production of the 224 amino acid protein, retinoschisin-1 (RS1) (1). RS1 is primarily secreted by photoreceptors and bipolar cells, and influences cell-cell interactions through a ubiquitous discoidin domain, facilitating photoreceptor-bipolar cell signaling, and stabilizing the extracellular environment of the retina (2, 3). In the absence of functional RS1, the laminar architecture of the retina becomes disrupted, causing retinal layer separation, known as schisis, and the formation of retinal cysts. Cysts, a hallmark of XLRS, spatially divide the inner nuclear and outer plexiform layers, separating the bipolar cells and photoreceptors. This separation impedes electrical transduction in the retina, leading to a characteristic waveform in electroretinography (ERG) termed an electronegative ERG, in which the ascending b-wave does not exceed the baseline.

From a clinical perspective, almost all patients with XLRS experience foveal involvement, impairing central vision (4, 5). Comparatively, around 50 percent of patients also exhibit peripheral involvement (2, 4). The progressive impairment of central vision due to macular atrophy can result in substantial functional vision loss. Additionally, the development of schisis significantly increases the risk of vitreous hemorrhage and full thickness retinal detachment following even mild ocular trauma (4). Strategies to manage cyst severity are needed.

Currently, Carbonic Anhydrase II Inhibitors (CAIs) serve as the primary medical therapy available for patients with XLRS. The proposed mechanism of CAIs involves inhibition of membrane-bound carbonic anhydrase (CA) in the retinal pigment epithelium (6). Fluid transport from the subretinal space into the choroid is enhanced experimentally following introduction of a carbonic anhydrase inhibitor (CAI), acetazolamide, suggesting CA is involved in maintaining fluid homeostasis of the retina (6). However, despite their efficacy in reducing intraretinal cyst formation in some patients, systemic CAIs are associated with well-known systemic side effects affecting the endocrine, gastrointestinal, cardiovascular, and central nervous systems, while there is often poor compliance with topical CAIs, especially in children (7-9).

While CAIs have shown short term benefits including mild decrease in central macular thickness, reduction of intraretinal cyst formation, and improved vision, it is not known whether they prevent the long-term progression of XLRS (8). Additionally, several studies have reported limited effects on retinal morphology or functional benefit, leading to inconsistent use of CAIs across different treatment centers (5, 10).

Sudden discontinuation of topical brinzolamide, a commonly used CAI in clinical practice, can lead to increased cyst formation and elevated intraocular pressure during periods of drug holiday (8, 9). There are currently no studies demonstrating long-term efficacy or safety, and CAIs are at best a treatment with variable efficacy, not a cure.

Given the lack of effective long-term therapy, ongoing efforts in gene therapy have emerged as a potential avenue for treatment. A Phase I/IIa trial was conducted on human subjects which involved a single intravitreal injection of an AAV8 vector carrying the RS1 gene (11). This safety study was introduced after good success in a mouse model; however, in human subjects, several ocular inflammatory outcomes were recorded (11). An additional human study utilizing an AAV2 vector to deliver intravitreal RS1 also reported inflammatory outcomes (12).

Compared to intravitreal injections delivering equivalent doses of AAV, subretinal injections elicited lower levels of humoral immune reactions both in mice and in primates (13). In this study, whether a subretinal injection of low-dose adeno-associated virus, AAV2/4-EF1α-RS1, can rescue the retinal phenotype with reduced risk of vector toxicity and ocular inflammation was investigate. In this study, a low dose AAV2/4-EF1α-RS1 gene therapy was subretinally administered to Rs1-KO mice, a model for XLRS. To account for the potential effect of the subretinal injection procedure, a control group receiving a subretinal injection of a hypertonic buffer, similar in osmolarity to the AAV storage buffer, was included.

Subretinal injections of low dose AAV2/4-EF1α-RS1 was investigated for correcting the retinal phenotype in the Rs1-KO mouse detectable by electroretinography (ERG), optical coherence tomography (OCT), and a visually guided swim assay (VGSA). Unexpectedly, results revealed a therapeutic effect of the buffer, including cyst reduction accompanied by long-term ERG improvement, constituting a novel finding in XLRS.

### Example 1 Example 1 Hypertonic Buffer Alleviates Intraretinal Cysts, Promotes Survival of Cone Photoreceptors, and Preserves Retinal Function in a Mouse Model of XLRS Methods

As described in detail below, Rs1-KO mice were subretinally injected with an AAV2/4 vector (n=10) containing the RS1 gene driven by an Ef1α promoter, a hypertonic buffer (n=15) (180 mM NaCl, 0.001% F68/PBS (pH 7.4)), or isotonic buffer (n=7) (155.2 mM NaCl, 0.001% F68/PBS, pH 7.0). A sham puncture group was also included (n=6). Endpoints included electroretinogram (ERG), optical coherence tomography (OCT), a visually guided swim assay (VGSA), and immunohistochemistry.

### 1. Study Design

Natural history, as well as gene therapy efficacy and effect of subretinal buffer injections, were evaluated in a mouse model of juvenile X-linked Retinoschisis (Rs1-KO). The natural history of this Rs1-KO mouse model has been reported, including the variable expressivity of the phenotype (14). Treatment-naïve eyes from postnatal day (p) 15- to 12-months of age were examined. OCT images were analyzed for both outer nuclear layer (ONL) thickness and cyst severity.

To study the effect of gene therapy, eyes treated with a subretinal injection of gene therapy vector (AAV2/4-EF1α-RS1) (n=10) were compared to hypertonic buffer-injected eyes (n=15) and treatment-naïve eyes (n=30). The hypertonic buffer-injected cohort was initially included to account for the high salt content solution the vector is produced and stored in as part of standard vector formulation and prevention of viral clumping. Injections alternated between the left and right eyes to avoid systematic bias. The selection of an AAV2/4 vector was based on a previously demonstrated tropism for all layers of the retina, including the photoreceptor and bipolar cell layers, the primary secreters of RS1 (15).

Injections were performed between p24 and p31. Outcome measures include ERG, OCT, VGSA, and immunohistochemistry. ERG was performed at 1-, 2-, 3-, and 5-months post injection (MPI) to assess retinal function. OCT was performed at 2- and 3-weeks post-injection (WPI), then 1-, 2-, 3-, and 5 MPI to assess cyst severity and retinal structure. A visually guided swim assay was performed at 5 to 6 MPI to assess functional vision. The eyes were collected and fixed at 6 MPI for immunohistochemistry. For the purpose of ERG and OCT experiments, one eye is considered a biological data point. For the purpose of the VGSA, each data point is representative of the averaged time-to-platform of a mouse for 20 different trials using randomly selected platform positions. Mice were treated in one eye with either vector or hypertonic buffer. Mice treated with gene therapy in one eye and hypertonic buffer in the fellow eye were excluded from the VGSA. Both male (Rs1 hemizygous mutants) and female knockout mice (Rs1 homozygous mutants) were used in this study.

To investigate the effect of the buffer, the subretinal injection of the hypertonic buffer was compared to the subretinal injection of an isotonic buffer and a sham puncture injection group that included a mechanical puncture of the sclera without deposition of a subretinal buffer fluid. Endpoints included ERG and OCT, and the same timelines as stated above were used in this group.

### 2. Animal Husbandry and Ethics Statement

This study was performed in accordance with the recommendations set forth by the National Institute of Health in the Guide for the Care and Use of Laboratory Animals. Animals were handled in accordance with the approved Institutional Animal Care and Use Committee (IACUC) protocol #4031421 at the University of Iowa. The Rs1-KO C57Bl/6J mouse model was kindly supplied by Paul Sieving, M. D., Ph.D. at the National Eye Institute. Generation of the Rs1-KO mouse model was previously described (16). This mouse model contains a deletion of exon 1 and a 1630 bp fragment of intron 1 on the Rs1 gene (16).

Animals were housed according to the IACUC recommendations. Humane endpoints were observed, and the method of euthanasia used was carbon dioxide inhalation followed by cervical dislocation.

### 3. Statistical Analysis

Analysis was performed using GraphPad PRISM 10.0 (GraphPad Software, Inc, San Diego, California, USA). Two-way ANOVA was performed for ERG and OCT metrics and followed by multiple comparisons (Tukey's test, non-parametric). One-way ANOVA was performed for VGSA and cone density followed by multiple comparisons (Tukey's test, non-parametric). Simple linear regression was used for correlation of peak cyst area vs 5 MPI ERG amplitudes.

### 4. Genotyping Information

Genotyping was performed using Taq Polymerase (New England biolabs, Ipswich, MA) using the primers listed in Table 1. The PLA2 primer was previously described (16). A 10 microliters (μL) reaction volume was used containing 2 μL of DNA (approximately 25 μg/μL), 3.7 μL of ultrapure water, 1 μL of 10× Buffer/Mg++, 1 μL of 20 mM primer mix (Table 1), 0.2 μL of 50 mM dNTPs, 0.1 μL of rTaq, and 2 μL of 5× Betaine. Cycling conditions are as follows; initial denaturing at 94° C. for 30 seconds, annealing at 57° C. for 30 seconds and extension at 72° C. for 30 seconds, with a final extension step at 72° C. for 4 minutes. This produces a 516-base pair (bp) wild-type band and a 300 bp knockout band.

TABLE 1
Primers

| | |
|---|---|
| RS1 Promoter | 5′-TAGGGGCCCACATCTTCCAAC-3′ |
| F2 33% | (SEQ ID NO: 1) |
| PLA2 33% | 5′-GTTCTTCGGACGCCTCGTCAACAC-3′ |
| | (SEQ ID NO: 2) |
| RSWT2-R 33% | 5′-GTGACAAAGAGCCACACAACAGTGACC-3′ |
| | (SEQ ID NO: 3) |

### 5. AAV Packaging

The human RS1 gene was cloned into a shuttle plasmid pFBAAV and provided by the Viral Vector Core at the University of Iowa. The bacterial backbone is from Invitrogen's pFastflac system. The shuttle plasmid consists of the pFBAAV backbone, an EF1α promoter, and a bovine growth hormone polyadenylation signal. The plasmid was packaged into an AAV2/4 capsid by the Budd Tucker Laboratory at the University of Iowa. The AAV was formulated at 2×10⁹ viral genomes (vg)/mL titer, and the composition of the storage buffer is listed in Table 2. Titering was performed using digital droplet PCR.

TABLE 2
Buffer solutions

| | |
|---|---|
| AAV storage | 180 mM NaCl |
| buffer | 10 mM Na3PO4 |
| | UltraPure water |
| | pH 7.2-7.4 |
| hypertonic | 0.001% Gibco Pluronic F-68 |
| injection | Gibco dPBS (−/−) (Catalog #14190144) |
| buffer | [Contains 155.2 mM NaCl] |
| | Additional 24.8 mM NaCl |
| | (for 180 mM NaCl total) |
| | pH 7.4 |
| isotonic | 0.001% Gibco Pluronic F-68 |
| injection | Gibco dPBS (−/−) (Catalog #14190144) |
| buffer | [Contains 155.2 mM NaCl] |
| | pH 7.4 |

### 6. Subretinal Injection

Mice were anesthetized using a ketamine/xylazine mixture (87.5 mg/kg ketamine, 12.5 mg/kg xylazine). A tropicamide ophthalmic solution of 1% was applied three minutes prior to injection to dilate the eyes. Proparacaine was applied as a topical anesthetic and 10% povidone-iodine was applied as a topical antiseptic. A partial thickness puncture was made just posterior to the limbus through the sclera with a 30-G sharp needle, then a 33-G blunt Hamilton needle was inserted and positioned between the RPE and the retina. Two μL of either gene therapy vector at a concentration of 2×10⁹ vg/mL, a hypertonic buffer, or an isotonic buffer was injected into the subretinal space (formulation available in Table 2). The injection of a solution subretinally creates a separation of the retina and the RPE known as a subretinal bleb. This bleb was assessed by the injector as seen through the operating microscope and in the rare cases with no visible bleb or a large vitreous hemorrhage, the mouse was excluded. For the sham injection puncture experiment, the same sharp needle was used to make an entry into the eye, then the blunt Hamilton needle was introduced subretinally however no liquid was delivered. Mice with retinal detachments that persisted until the first OCT time point were excluded. The mouse was then given topical ophthalmic ointment consisting of neomycin, polymyxin B sulfates, bacitracin zinc, and hydrocortisone. Mice were injected with atipamezole to aid in recovery from the anesthetic.

### 7. Electroretinogram

Dark adaptation occurred overnight, and ERGs are conducted in the morning to control for potential diurnal variations. Mice were anesthetized using a ketamine/xylazine mixture (87.5 mg/kg ketamine, 12.5 mg/kg xylazine). A tropicamide ophthalmic solution of 1% was applied to the eyes for pupil dilation. A 2.5% Hypromellose solution (Akron, Lake Forest, Illinois) was applied to maintain corneal hydration during testing. Testing was performed on the Diagnosys Celeris with integrated heated platform (Diagnosys, Lowell, MA). Electrodes were placed on the cornea of bilateral eyes, and impedance was kept below 10 kΩ. Eyes were exposed to various light intensities in a modified ISCEV protocol (17). To evaluate rod electrical function in treated and untreated Rs1-KO mice, animals were dark-adapted (DA) overnight to isolate the rod response, and eyes were subjected to 15 flashes of 0.01 cd·s/m² light (0.01 dim flash) then 15 flashes of dark-adapted 3.0 cd·s/m² standard combined response (3.0 SCR). To evaluate cone electrical function in treated and untreated Rs1-KO mice, eyes were light-adapted (LA) for 10 minutes to bleach the responses from rods, and subjected to 15 flashes of 3.0 cd·s/m² light (3.0 flash) followed by 20 flashes of a 5 Hz flickering light of 3.0 cd·s/m² (5 Hz Flicker), which is more reproducible in mice than the standard light-adapted 30 Hz flicker in humans (18). Multiple comparisons were performed between every group at each time point. Non-significant comparisons were not graphed.

### 8. Optical Coherence Tomography

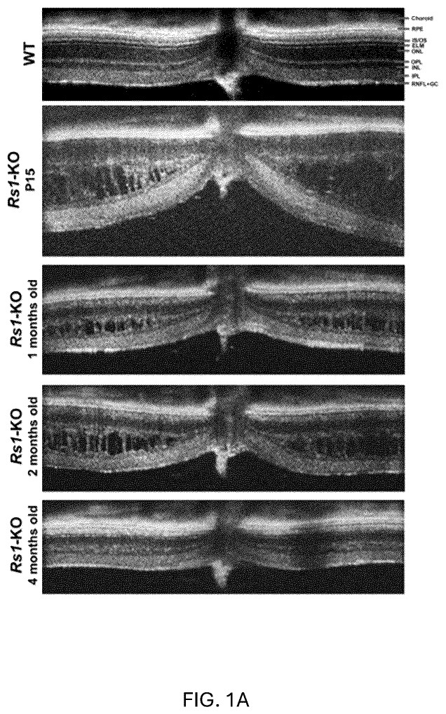

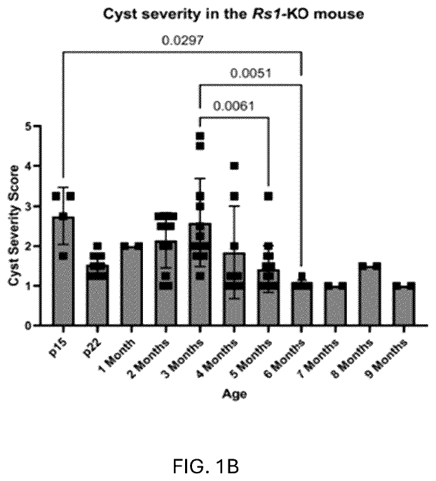

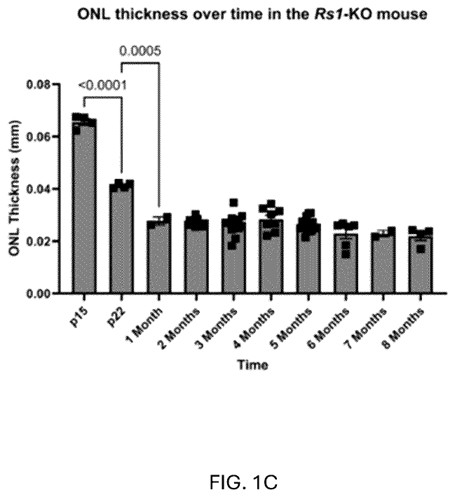

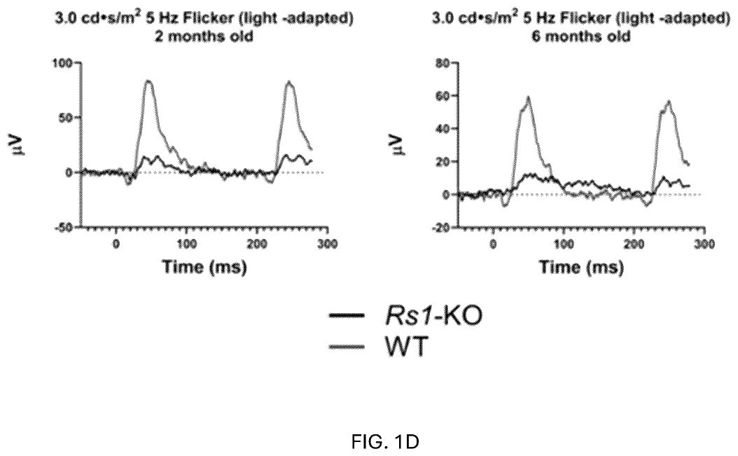

Mice were anesthetized using a ketamine/xylazine mixture (87.5 mg/kg ketamine, 12.5 mg/kg xylazine), and Tropicamide ophthalmic solution 1% was applied for pupil dilation. 1% carboxymethylcellulose was applied to maintain corneal hydration during testing. OCTs were conducted in the afternoon to control for potential diurnal variations. Cyst severity in the natural history data (FIGS. 1A-1) was determined via a modified scoring protocol from previously described literature (19, 20). Briefly, measurements of cyst height were taken at four points equidistant, 500 μm from the optic nerve, two on the superior-inferior line bisecting the optic nerve, and two on the nasal-temporal line bisecting the optic nerve. Measurements were then translated to a scoring scale as previously described, in which a score of 1 was assigned to zero cyst height and a score of 6 was assigned to cyst height >100 μm. The score was averaged across all four points (20).

For all other cyst severity studies, volumetric scans were taken centering on the optic nerve along with nasal and temporal scans. Cyst severity was determined via manual segmentation of cyst area using the Image J software. Center scans, those which best bisect the optic nerve, were quantified by manually segmenting individual cyst cavities and recording the total area of cysts in the scan. Similar quantifications were performed on nasal-temporal slices located approximately 500 μm superior and inferior of the center scan, and area was reported as a sum of these measurements. Multiple comparisons were performed between every group at each time point. Non-significant comparisons were not graphed.

### 9. Visually Guided Swim Assay

AAV2/4-EF1α-RS1 vector-treated mice and mice injected with the hypertonic buffer underwent a VGSA performed in light and dark conditions to assess functional vision at approximately 5 to 6-months of age compared to treatment-naïve controls. The VGSA has been described in detail elsewhere (21). In brief, the mice undergo four training days in the light, four testing days in the light, two training days in the dark, and four testing days in the dark, and the performance of each mouse to 5 different platform locations were measured during each training or testing day. For each trial, the mice were placed into a plastic pool and were expected to find a platform. Five of the eight possible platform positions are randomly assigned each day with all platforms being used approximately the same number of times. The maximum time to platform (TTP) was set at 60 seconds for each trial to prevent fatigue and undue stress. After the mouse had completed the trial or after 60 seconds of searching, the mouse was removed from the pool, dried, and placed in its cage until the next trial. Mice were excluded in rare cases where they could not be motivated to participate and search for the platform. Data from all trials was excluded if a mouse had a swim time greater than one standard deviation from the mean of the group or consistent floating occurred, such as more than three episodes of floating per trial or more than three corrections required per trial. Corrections for floating include snapping fingers or touching the mouse's tail.

### 10. Immunohistochemistry

Eyes were enucleated and a small puncture was made through the cornea with a 27-G needle. Eyes were placed in 4% paraformaldehyde for 12 hours and transferred to PBS for 24 hours. After fixation, eyes were embedded in Tissue-Tek O.C.T. compound (VWR, Batavia, IL) and frozen in a bath of 2-methylbutane cooled with liquid nitrogen. Eyes were sectioned on the superior-inferior axis at a thickness of 10 μm using a Cryostat microtome and stored at −80° C. for future use.

For immunohistochemistry, sections were permeabilized using 0.3% Triton X-100 in PBS (buffered at pH 7.4) for 10 minutes at room temperature, blocked with a blocking buffer containing 5% BSA, 5% normal goat serum, and 0.05% Triton X-100 in PBS for 1 hour at room temperature, and then incubated with primary antibodies or biotinylated-peanut agglutinin (biotinylated-PNA) in a dilution buffer containing 5% BSA, 1% normal goat serum, and 0.05% Triton X-100 in PBS at 4° C. overnight. The following day, samples were washed in 1×PBS three times before incubation with secondary antibodies or streptavidin Alexa Flour-568 conjugate at room temperature for 1 hour. After washing in 1×PBS an additional three times, samples were mounted with VectaShield mounting medium with DAPI (Vector Laboratories, Burlingame, CA). Images were taken using a fluorescence microscope and color channels were overlayed in Image J. No contrast enhancements or brightness levels were altered after acquisition.

Antibodies and reagents used for immunohistochemistry were as follows: anti-CtBP2/RIBEYE antibody (BD Transduction Laboratories #612044; 1:200 dilution); biotinylated-PNA (Vector Laboratories #B-1075; 1:500 dilution).

Images were taken directly superior and inferior of the optic nerve head and were gathered at 40× magnification. Three serial images were acquired on either side of the optic nerve leading to 6 total images gathered per eye. These images were then independently quantified by three individuals masked to treatment groups and the number of cones per image was averaged. The quantifications were averaged and divided by the image width to produce the reported average cones per 100 m.

For immunohistochemistry on the whole flat-mount retinas, the retina was permeabilized using 0.3% Triton X-100 in PBS for 10 minutes at room temperature, blocked with blocking buffer containing 5% BSA, 5% normal goat serum, and 0.05% Triton X-100 in PBS at 4° C. overnight. The following day, samples were washed in 1×PBS two times for 10 minutes in room temperature before incubation with secondary antibody streptavidin Alexa Flour-568 conjugated and Hoechst 1:1000 (ThermoFisher Hoechst 33342) at room temperature for 1 hour tilted in a 2 mL eppendorf tube. After washing in 1×PBS two times for 10 minutes at room temperature, samples were mounted with Fluoromount-G (Electron Microscopy Science #17984-25). Images were taken on a THUNDER Imager (Leica DM6B microscope equipped with a Leica DFC9000 GT camera), z-stacks were between 10-17 stacks at 1.5 μm and computational clearing of z-stacks was carried out using LASx software. With the use of ImageJ, two rectangle boxes per retina were cropped to approximately 350×350 μm to obtain a uniform area in which to count cones on a flat mount. The image was converted to 8-bit and thresholded to isolate the cone staining on the image. Once these steps were completed, a particle analysis was conducted to count and label selected cones present in the cropped image. Cone density of buffer-injected or sham-punctured eye was normalized to its untreated fellow eye and reported as a percentage.

### Results

### 1. Natural History of Disease in the Rs1-KO Mouse Compared to WT BL/6 Mouse.

The features of the Rs1-KO mouse model were characterized. Cyst severity was quantified over time in the Rs1-KO mouse using a modified cyst severity scoring system, as previously described by Bush et al. (19). FIGS. 1A-1D demonstrate typical OCT findings in the Rs1-KO mouse at p15, 1-, 2-, and 4-months of age, compared to a WT mouse (FIG. 1A). Retinal cysts can be observed in the Rs1-KO mouse retina up to 3 months of age, in contrast to the organized, laminar retinal architecture of the WT mouse. Cysts are apparent as early as p15 with an average severity score of 2.75 out of 6 (FIG. 1B). Cysts appear the most severe at 2- to 4-months old, hitting a local maximum at 3-months. Thereafter, cyst severity reduced with maturity.

At p15, Rs1-KO mice have comparable ONL thicknesses (65.56 μm) as mice that are WT (62.8±6.23) (22), or heterozygous for the Rs1 gene. In untreated Rs1-KO mice, significant thinning of the ONL occurred from p15 to p22 (FIG. 1C, p<0.0001), during which the ONL thickness was decreased by more than 37%. After 1-month, the ONL thickness in Rs1-KO mice did not significantly change, indicating that most of the photoreceptor cell loss occurred during eye maturation in this mouse model.

ERGs measure the electrical response of the retina in vivo to understand how the disease affects signal transmission in light and dark conditions. ERGs of Rs1-KO mice consistently demonstrated a reduction in function of rod-dependent and cone-dependent visual pathways compared to WT eyes. FIGS. 1A-1D demonstrates typical ERG waveforms for this XLRS model at 2 and 6 months of age under light-adapted (5 Hz flicker) metrics (FIG. 1D). In summary, features of the Rs1-KO mouse model resemble the clinical course in humans, including cyst formation, ONL thinning, and diminished electrical activity of retinal photoreceptors and bipolar cells observable on ERG. The phenotypes observed mirror those reported by Sieving et al. (14).

By administering gene therapy at 1-month of age, we aimed to determine if the intervention could mitigate the severity of cysts in the retina and subsequently improve the electrical signaling of retinal cells after the initial ONL thinning phase. Results from vector-treated and hypertonic buffer-injected eyes were compared to those of treatment naïve eyes (untreated).

### 2. Cyst Severity is Significantly Reduced in Vector-Treated and Hypertonic Buffer-Injected Eyes at 1 MPI.

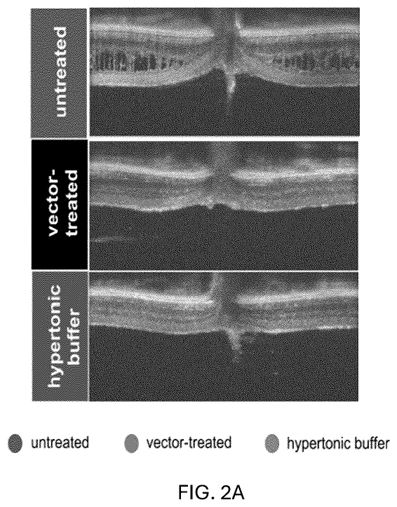

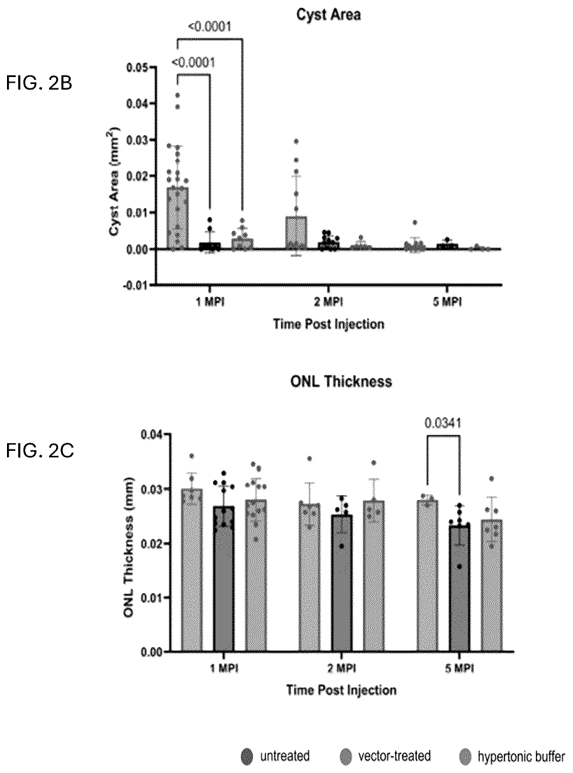

Using cyst area (mm²) of central OCT cross-sections as a measure of cyst severity, we observed that cysts were nearly completely ameliorated in vector-treated Rs1-KO eyes (n=10) and hypertonic buffer-injected Rs1-KO eyes, in contrast to untreated eyes at 1 MPI (vector-treated: 0.001871 mm², p<0.0001; hypertonic buffer: 0.002878 mm², p<0.0001, untreated: 0.01691 mm², FIG. 2B). This reduction in cyst burden was particularly noteworthy because it coincided with the critical cyst developmental period around 2-months of age, a time when peak cyst formation is typically observed in Rs1-KO mice (FIGS. 1A-1D). This data suggests that the injection of either the vector or hypertonic buffer can effectively reduce cysts during a crucial phase of cyst development. Interestingly, there was no difference in the extent of cyst reduction between the vector and hypertonic buffer-injected eyes.

At subsequent time points, 2 and 5 MPI, since cyst severity naturally decrease in untreated Rs1-KO eyes (FIG. 1B), we did not observe significant differences in cyst severity among untreated, vector-treated, or hypertonic buffer-injected eyes (14) There is no notable change in ONL thickness from 1 to 5 MPI in all cohorts; however, at 5 MPI, vector treated eyes had a statistically thinner ONL than untreated eyes (p=0.0341) (FIG. 2C).

Cysts spatially separate bipolar cells and photoreceptors, and it is hypothesized that the lower b-wave amplitudes and/or electronegative ERG phenotype associated with XLRS is a consequence of this spatial separation. To examine the potential influence of reduced cysts on retinal electrical signaling, we conducted ERGs under light-adapted and dark-adapted conditions.

### 3. Hypertonic Buffer Outperforms Untreated and Vector-Treated Eyes in Light-Adapted ERGs, but not in Dark-Adapted ERGs.

ERGs were performed at 1, 2, 3, and 5 MPI to evaluate treatment efficacy, and ERG amplitudes in Vector-treated Rs1-KO eyes (n=10) or hypertonic buffer-injected Rs1-KO eyes (n=15) were compared to those in their contralateral untreated Rs1-KO eyes.

### Light-Adapted

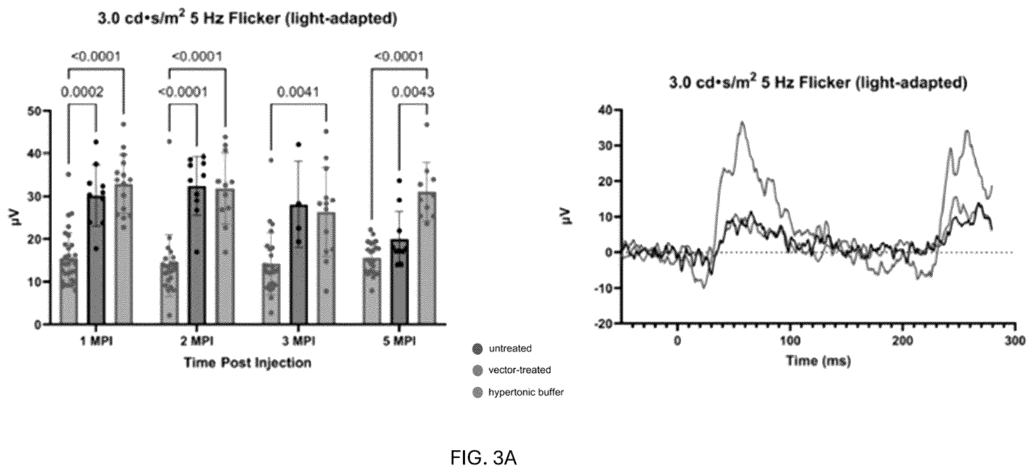

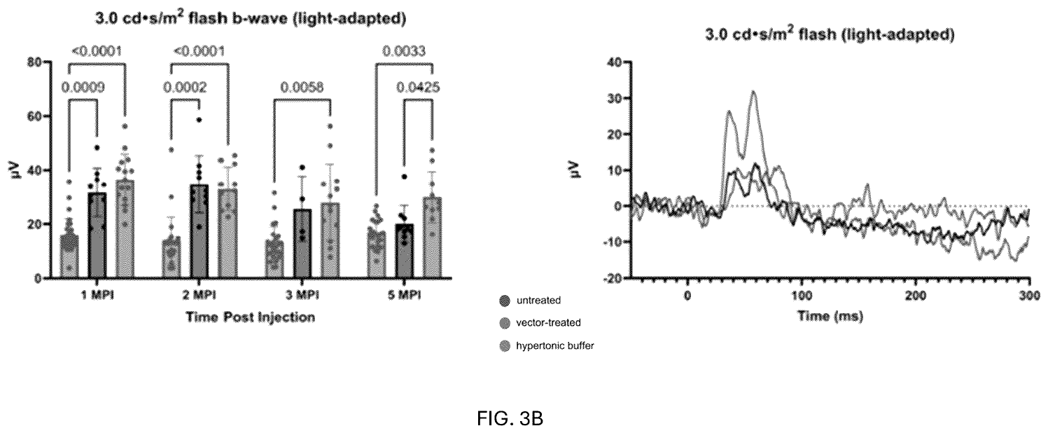

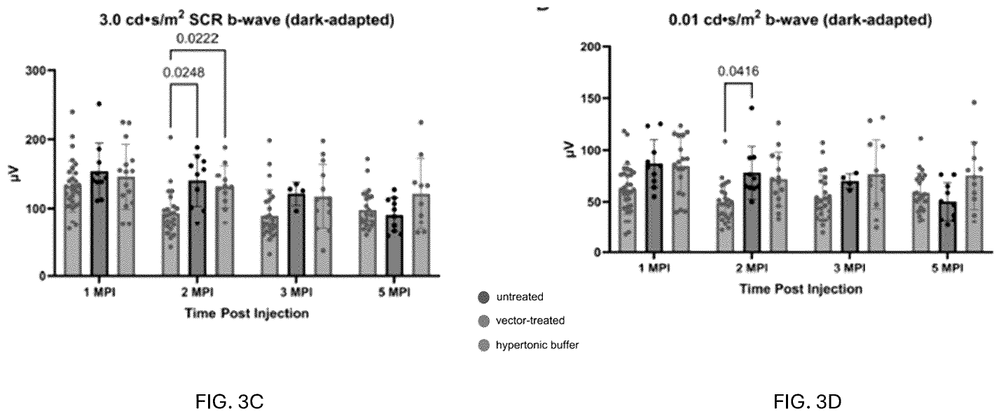

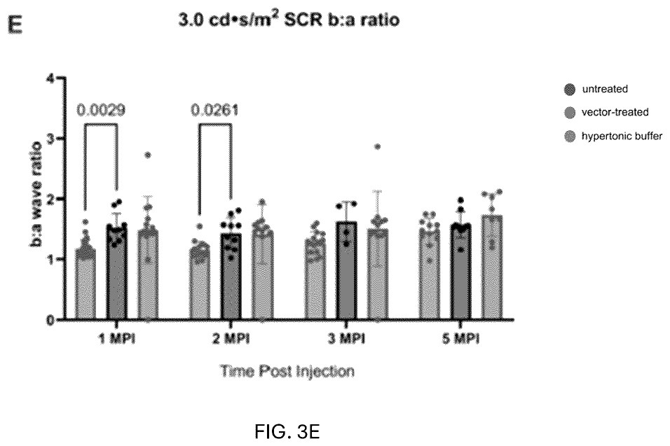

The retinal cone pathway was assessed using two light-adapted ERG assays: the 5 Hz flicker and the 3.0 flash. At 1 MPI, both vector-treated and hypertonic buffer-injected eyes showed significantly higher amplitudes, suggestive of superior cone function, compared to untreated eyes in the 5 Hz flicker assay (vector-treated: 30.17±7.14 μV, p=0.0002; hypertonic buffer: 32.86±6.88 ρV, p<0.0001; untreated: 15.42±6.17 μV, FIG. 3A). This effect persisted until 2 MPI in vector-treated eyes (vector-treated: 32.42±6.89 μV, p<0.0001) after which amplitudes decreased and significance over untreated eyes was lost. Interestingly, hypertonic buffer injected eyes continued to have significantly higher amplitudes than untreated eyes to 5 MPI (hypertonic buffer: 31.05±6.82 μV, p<0.0001; FIG. 3A).

Notably, at 5 MPI, hypertonic buffer-treated eyes demonstrated significantly higher amplitudes than vector-treated eyes (19.98±6.49 μV, p=0.0043), indicating a more robust and sustained rescue of cone function in the hypertonic buffer group compared to the low-dose AAV2/4-EF1α-RS1 vector (FIG. 3A). This data suggests that subretinal injection of the hypertonic buffer provides a more effective long-term improvement in cone function than the vector treatment.

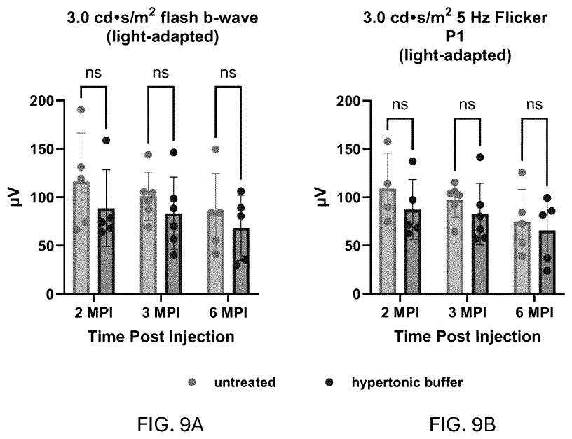

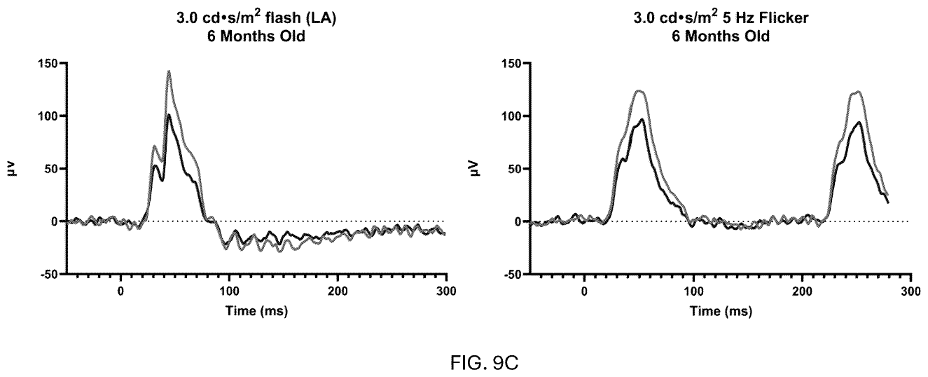

Similarly, in the 3.0 flash assay, vector-treated eyes demonstrated significantly higher amplitudes, suggestive of superior cone function, compared to untreated eyes until 2 MPI (vector-treated: 34.70±10.56 μV, p=0.0002; hypertonic buffer: 33.04±8.01 μV, p<0.0001; untreated: 13.75±8.85 μV, FIG. 3B) after which time amplitudes began to decrease. However, hypertonic buffer-injected eyes exhibited a more robust and long-term improvement in function with higher amplitude ERGs persisting to 5 MPI (29.93±9.57 μV, p=0.0033), when compared to untreated eyes (16.43±5.19 μV), and even demonstrated significantly higher amplitudes than vector-treated eyes at 5 MPI (20.03±6.87 μV, p=0.0425). This robust and sustained improvement in cone function observed in the hypertonic buffer group was an unexpected finding, indicating that the subretinal injection of the buffer alone enabled long-term benefits in retinal cone function. This phenomenon is specific to JXLR, as injection of a hypertonic buffer into eyes without XLRS typically reduces the ERG amplitudes (FIGS. 9A-9C). Since injection of buffer alone causes an increase in b-wave amplitudes in the absence of restoring RS1 protein expression, this phenotype could be the consequence of cyst amelioration. Representative waveforms demonstrate these findings at 5 MPI (FIGS. 3A-3B).

### Dark-Adapted

To measure the electrical function of the eye in dark conditions, the 3.0 SCR and 0.01 dim flash ERG assays were utilized. At 1 MPI, no significant difference was observed in the b-wave amplitudes between untreated eyes and vector-treated or hypertonic buffer-injected eyes in the 3.0 SCR (vector-treated: 153.8±40.87 μV, p=0.3211; hypertonic buffer: 146.0±47.17 μV, p=0.5819, untreated: 132.0±37.00 μV, FIG. 3C), or 0.01 dim flash assays (vector-treated: 86.91±23.39 μV, p=0.0691, hypertonic buffer: 84.48±27.73 μV, p=0.0541, untreated: 62.01±24.05 μV, FIG. 3D). At 2 MPI, both the vector-treated and hypertonic buffer-injected eyes showed significantly higher amplitudes, suggestive of an improved combined function of rod and cone photoreceptors in the 3.0 SCR (vector-treated: 140.2±37.56 μV, p=0.0248, hypertonic buffer: 131.3±30.43 μV, p=0.0222, untreated: 93.07±32.97 μV, FIG. 3C). A similar effect was seen at 2 MPI in vector-treated eyes during the 0.01 dim flash assay (vector-treated: 78.12±25.75 μV, p=0.0416, FIG. 3D). Under the 3.0 SCR b:a wave ratio, vector treated eyes had significantly higher b:a wave amplitude ratios than untreated eyes util 2 MPI (vector-treated: 1.43±0.26 μV, p=0.0261; untreated: 1.16±0.14 μV, FIG. 3E). However, these improvements were transient and were not maintained at 3 and 5 MPI. These results indicate that injection of low-dose AAV2/4-EF1α-RS1 vector or hypertonic buffer did not have a notable long-term effect on rod-dependent retinal function, nor on rod-cone combined function as measured by the 3.0 SCR test. Improvements in ERG amplitudes after the injection of hypertonic buffer were most notable in cone-dependent ERGs.

Following the observed improvements in cone specific electrical pathways, whether these improvements were associated with differences in functional vision was investigated.

4. Vector-Treated and Hypertonic Buffer-Injected Mice Perform Faster in a Light-Adapted Swim Assay than Untreated Rs1-KO Mice.

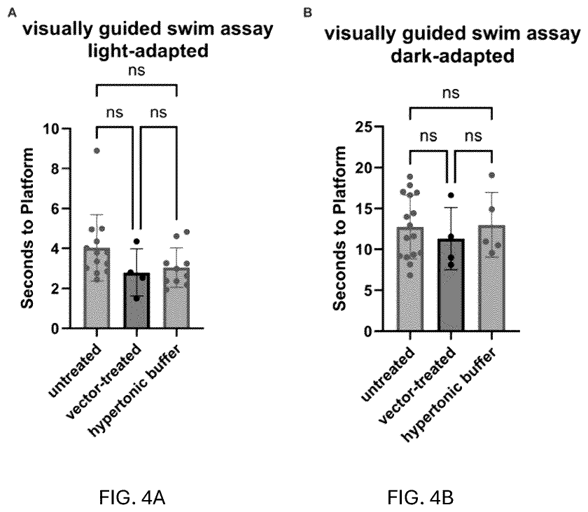

Functional vision in mice subretinally injected with the low-dose AAV2/4-EF1α-RS1 vector or hypertonic buffer, as well as the untreated Rs1-KO mice, was measured using a VGSA. Mice between 5 to 6-months of age were placed in a pool and platform locations were randomly determined for each trial in light- and dark-adapted environments. One datapoint represents the averaged time it took for a mouse to reach the platform over 20 different trials using randomized platform positions. Administration of the vector or buffer was unilateral. In light-adapted conditions, we observed that vector-treated mice (2.80 seconds) and hypertonic buffer-injected mice (3.05 seconds) had a generally faster trend in average swim performance than untreated mice (4.04 seconds) in lighted environments (FIG. 4A). These results confirm that the subretinal injection of vector or buffer alone has potential to benefit functional vision in the light, which correlates with the apparent reduction of cysts, and improved cone pathway ERG signaling. The trends in improvement of functional vision seen in vector-treated mice was comparable to that observed in buffer-injected mice in light-adapted testing conditions. There was less of an impact on functional vision in dark-adapted swim environments (FIG. 4B).

Together, the long-term ERG data and functional vision assays suggest that subretinal injections of a hypertonic buffer solution alone led to significant reductions in cyst severity and cone-specific improvements in retinal function and functional vision. The improvements observed in buffer-treated eyes were comparable to, and in some instances superior to, those observed in vector-treated eyes, indicating that treatment with a low-dose AAV2/4-EF1α-RS1 was not efficacious. The reason behind the functional rescue observed in Rs1-KO mice subretinally injected with buffer is not clear.

### 5. Investigation of the Effect of Tonicity on Cone Rescue.

The injection of hypertonic buffer alone leads to significant improvements in cone-specific ERG amplitudes, light-adapted visual function, and retinal architecture in Rs1-KO mice. Surprisingly, the observed improvements in ERG amplitudes after buffer injection persisted for at least 5 MPI. Previous studies have demonstrated a temporary reduction in existing retinal cavities following the subretinal injection of a balanced salt solution; however, the long-term effects on visual function and electrical signaling of the retina has not been studied (23). Additionally, cystoid retinal conditions in XLRS patients can be modified by the application of diuretic ophthalmic solutions such as brinzolamide and acetazolamide but the connection between fluid reabsorption and cyst resolution is not well understood (8). Previous literature has suggested that the RS1 protein physically interacts with Na/K-ATPase and may act to mediate Na/K-ATPase activity, affecting signaling and ion gradients within the retina (24, 25).

The buffer solution, used both to store the AAV vector and used in the initial buffer injections, contained a high salt content and a greater than physiologic osmolarity. Therefore, we hypothesize that the tonicity of the injected buffer plays a role in promoting cyst resolution and improving retinal signaling. This hypothesis was investigated through a subretinal injection of an isotonic buffer. The isotonic buffer has a similar composition to the hypertonic buffer except possessing a lower concentration of NaCl. Additionally, to account for mechanical trauma to the eye during the injection procedure, a sham cohort was added and received a scleral puncture and introduction of a Hamilton needle subretinally, to mimic injection without the introduction of subretinal fluid, hereafter referred to as the “puncture” group. The endpoints of these two additional experimental groups also include ERG to measure retinal electrical function and OCT to measure retinal structure and cyst severity. The results for these tests are as follows.

6. Cyst Area is Reduced in Hypertonic Buffer and Isotonic Buffer-Injected Eyes, with More Robust Reduction Observed in the Hypertonic Buffer Group.

To determine if the tonicity of the injection buffer ameliorated cysts post injection, OCT was used to image the retinal layers in vivo. Cyst severity was evaluated by the segmentation of total cyst area in the central OCT scan. Outer nuclear layer (ONL) thickness measurements were taken to assess photoreceptor cell survival.

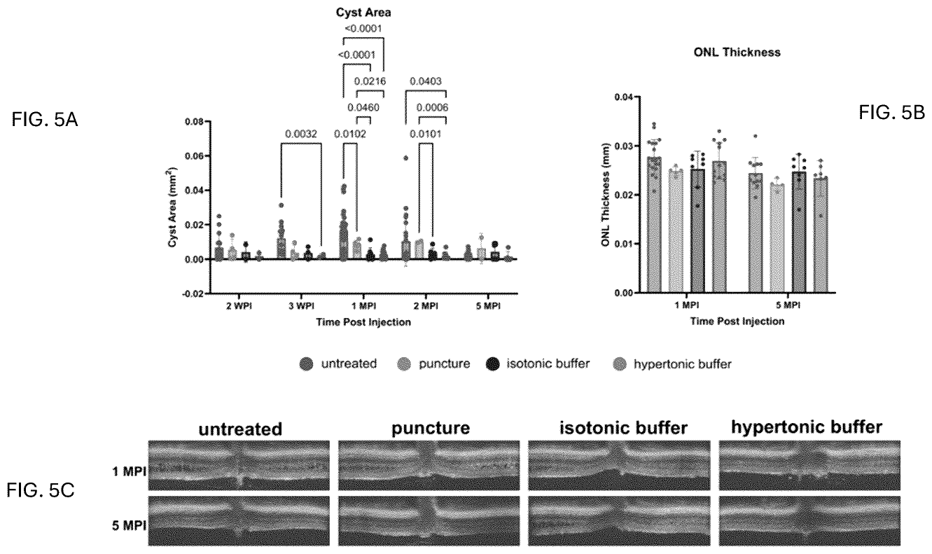

While all groups receiving buffer injections as well as the puncture group had reduced cyst severity at 3 weeks post injection (WPI), the group receiving injections of the hypertonic buffer had the greatest reduction (FIG. 5A). In the hypertonic buffer-treated group, cyst area was decreased by 83.77% compared to the cyst areas in untreated eyes (p=0.0032). Cyst areas were reduced by 68.32% and 69.53% in the puncture group and the isotonic group, respectively (FIG. 5A). While a sham puncture influenced cyst reduction, the greatest reduction was observed after the injection of the hypertonic buffer.

At 1 MPI, both hypertonic and isotonic buffer-treated eyes exhibited significantly less severe cysts than untreated or sham punctured eyes. In the hypertonic buffer-treated group, cyst area was decreased by 82.57% compared to the cyst area of untreated eyes (p<0.0001), and 65.46% compared to cyst area of sham punctured eyes (p=0.0216, FIG. 5A). This implies that subretinal injection of the buffer solution caused cyst reduction, and the reduction is not solely caused by the surgical puncture. In the isotonic buffer-treated group, cyst area was decreased by 82.53% compared to the cyst area of untreated eyes (p<0.0001, FIG. 5A), and 65.38% compared to cyst area of sham punctured eyes (p=0.0460, FIG. 5A). At 1 MPI, sham punctured eyes showed a 49.54% reduction in cyst area compared to untreated eyes (p=0.0102).

At 2 MPI, there is no longer a significant difference between cyst severity in the puncture group versus in untreated eyes, indicating that the alleviation of cysts in the scleral puncture group was transient. In contrast, the reduction in cyst severity persisted to 2 MPI in hypertonic buffer-injected eyes compared to untreated eyes (81.33% reduction, p=0.0403) and eyes that received only the sham surgical puncture (80.80% reduction, p=0.0006). At 2 MPI, isotonic buffer-treated eyes also demonstrated a significant reduction in cyst severity compared to sham punctured eyes (70.03% reduction, p=0.0101). Isotonic buffer-injected eyes did not have significantly reduced cyst severity compared to untreated eyes at 2 MPI (p=0.1145). Representative OCT images of all experimental groups at 1 and 5 MPI are demonstrated in FIG. 5C.

In summary, hypertonic buffer-injected eyes demonstrated an early and sustained reduction in cyst severity, starting as early as 3 WPI and persisting to 2 MPI. Additionally, the reduction in cyst severity was slightly greater in hypertonic buffer-injected eyes than in isotonic buffer-injected eyes at 3 WPI and 1 MPI.

Though cysts were decreased at early timepoints, ONL thickness was not different across time points between hypertonic buffer-injected, isotonic buffer-injected, sham punctured, and untreated eyes, suggesting that there is no difference in photoreceptor survival using this method.

7. Hypertonic Buffer-Injected Eyes have a More Robust Cone Rescue on Light-Adapted ERG Over Untreated, Sham Punctured, and Isotonic Buffer-Injected Eyes.

### Light-Adapted

Following an observed reduction in retinal cysts after subretinal injection of a hypertonic buffer, we investigated whether the improvement in cysts paralleled a similar improvement in electrical function of the retina.

Cone function was measured using two separate light-adapted ERG assays; the light adapted (LA) 3.0 flash and 5 Hz flicker.

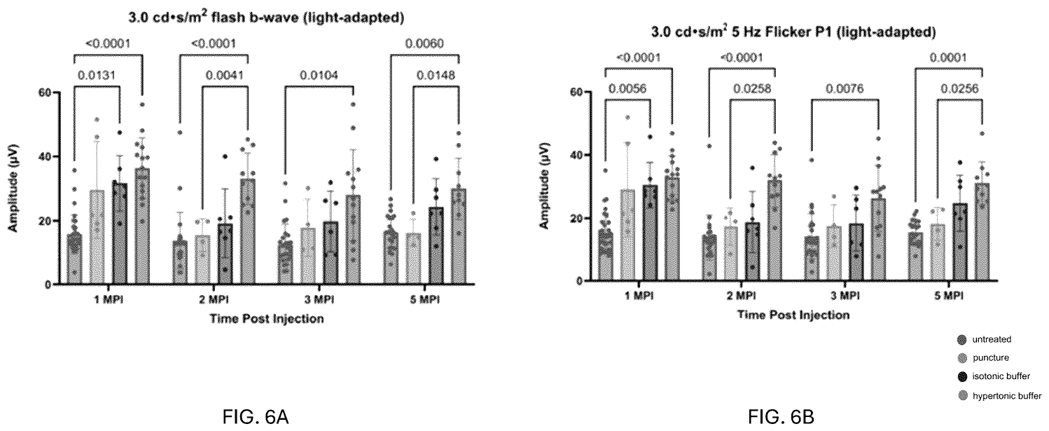

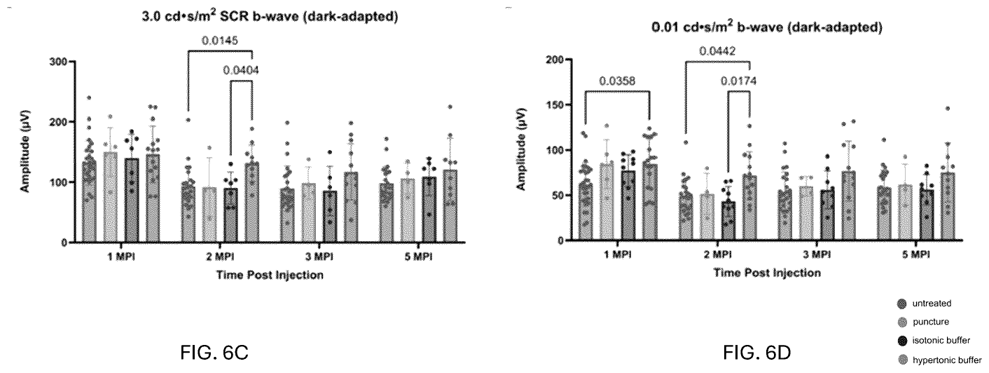

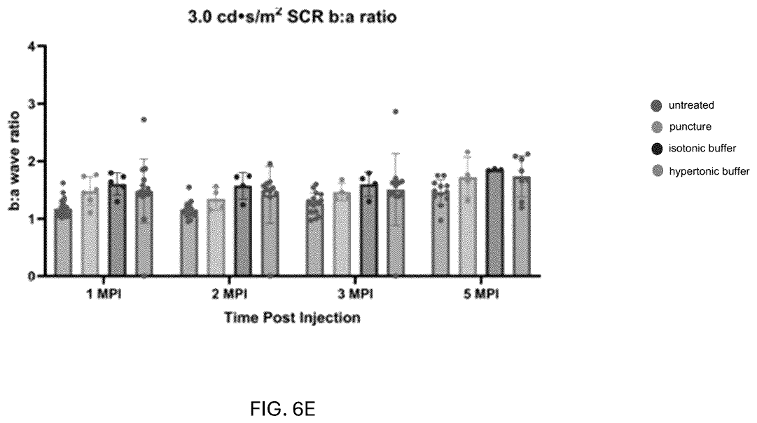

Under the light-adapted 3.0 flash assay, both hypertonic buffer and isotonic buffer-injected eyes showed significantly higher amplitudes, indicative of better cone cell functioning, compared to untreated eyes at 1 MPI (hypertonic buffer: 36.37±9.50 μV, p<0.0001; isotonic buffer: 31.62±8.71 μV, p=0.0131; untreated: 15.84±6.14 μV, FIG. 6A). At this time point, eyes that received the sham puncture also had increased amplitudes (29.59±15.10 μV). Hypertonic buffer-injected eyes continued to demonstrate improved cone functioning over untreated eyes throughout the study period, including at the study endpoint at 5 MPI (hypertonic: 29.93±9.57 μV, p=0.0060; untreated: 16.43±5.19 μV, FIG. 6A). At the endpoint, eyes that received puncture did not have a significant difference compared to untreated eyes, indicating that the effect of a surgical puncture on RS1 phenotypes was transient. Additionally, hypertonic buffer-injected eyes showed significant improvements over sham punctured eyes at 2 MPI (hypertonic: 33.04±8.01 μV, p=0.0041; sham punctured: 15.51±5.17 μV) and 5 MPI (hypertonic: 29.93±9.57 μV, p=0.0148; sham punctured: 16.21±4.29 μV, FIG. 6A).

Similar findings were observed in the second, cone-dependent assay, the 5 Hz flicker. Both hypertonic buffer and isotonic buffer-injected eyes showed significantly higher amplitudes compared to untreated eyes at 1 MPI (hypertonic buffer: 32.86±6.88 μV, p<0.0001; isotonic buffer: 30.43±7.19 μV, p=0.0056; untreated: 15.42±6.17 μV, FIG. 6B). However, the effect of isotonic buffer injection was not as notable as that observed in hypertonic buffer injected eyes, indicating the tonicity of the buffer could play a role in this observation. In contrast, hypertonic buffer-injected eyes continued to have significantly higher amplitudes, indicating sustained improved cone function, compared to untreated eyes until the study endpoint at 5 MPI (hypertonic buffer: 31.05±6.82 μV, p<0.0001; untreated: 15.52±3.71 μV, FIG. 6B). Additionally, hypertonic buffer-injected eyes had significantly higher amplitudes than sham punctured eyes at 2 MPI (hypertonic: 31.82±8.29 μV, p=0.0258; sham punctured: 17.36±5.93 μV, FIG. 6B) and 5 MPI (hypertonic: 31.05±6.82 μV, p=0.0256; sham punctured: 18.20±5.24 μV, FIG. 6B).

These results show that injection of a hypertonic buffer significantly improved the amplitudes of cone-dependent ERGs, and this effect persists even when cysts are naturally resolved due to age in the Rs1-KO mouse model. The injection of an isotonic buffer also had a beneficial effect, although the magnitudes of improvement were not comparable to those in hypertonic buffer-injected eyes. A transient effect was observed in eyes that received the scleral puncture only (without the injection of a buffer), indicating that the effect of surgical manipulation on cyst and retinal function was transient.

Together, these results show that the introduction of a subretinal buffer solution has unexpected beneficial effects on cyst severity and cone retinal function in eyes affected by XLRS, and the observed effect is long-term. Additionally, the tonicity of the injected buffer solution may play a role in the observed phenomenon.

### Dark-Adapted ERG

Combined rod-cone function was measured by the 3.0 SCR, and isolated rod function was measured using the 0.01 dim flash. In the 3.0 SCR, hypertonic buffer-injected eyes showed improved amplitudes over untreated (p=0.0145) and isotonic buffer-injected eyes (p=0.0404) at 2 MPI (FIG. 6C). In the 0.01 dim flash, hypertonic buffer-injected eyes showed improvements over untreated at 1 MPI (p=0.0358, FIG. 6D) and over both untreated (p=0.0442) and isotonic buffer-injected (p=0.0174) eyes at 2 MPI (FIG. 6D). Hypertonic buffer-injected eyes did not show improvements over sham punctured eyes in dark-adapted assays. There was no difference between experimental groups in the SCR b:a wave ratio (FIG. 6E). These findings suggest that hypertonic buffer-injection may lead to short term rescue of rods and that this effect may be influenced by the tonicity of the buffer.

8. Peak Cyst Severity on OCT is Negatively Associated with Cone Function on ERG at 5 MPI.

Hypertonic buffer-injected eyes had both the lowest cyst severity and the greatest ERG improvements in cone-dominant electrical pathways. To explore this finding, we investigated whether cyst severity is truly associated with a long-term impairment in electrical signaling of the retina. The correlation between peak cyst severity (the highest cyst area reached by the eye across different timepoints) and long-term retinal function (ERG amplitudes at 5 MPI) were analyzed, and included hypertonic buffer-injected, isotonic buffer-injected, sham punctured and untreated eyes. Generally, as part of the natural disease course in this XLRS mouse model, retinal cysts hit a local maximum of severity between 2- and 4-months of age (FIG. 1B). The presence of a negative correlation between peak cyst severity and 5 MPI ERG amplitudes would imply that the degree of cyst severity during critical times is correlated with long-term electrical function in the retina. By looking at long-term outcomes, we will determine if the reduction of cysts could influence long-term retinal function beyond any short-term benefits due to bringing the retinal layers closer together. Additionally, at 5 MPI there are little to no cysts present in untreated mice based on natural history and could not explain the variation in ERG amplitudes at that time.

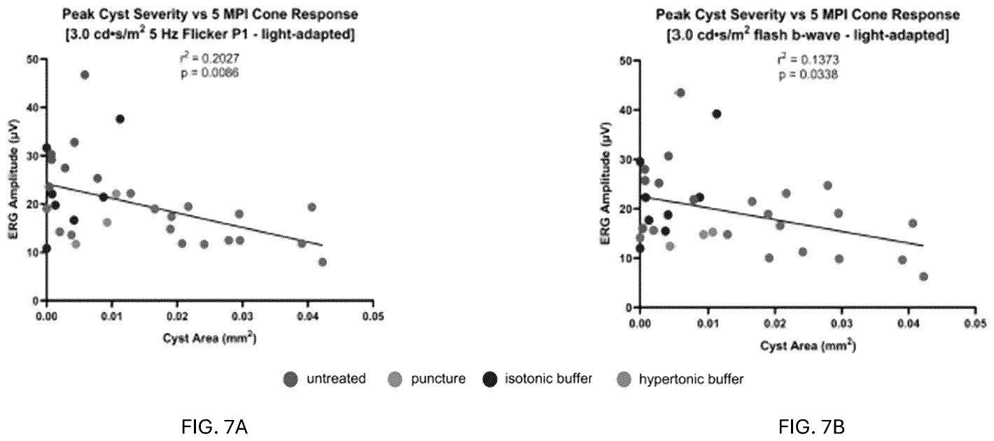

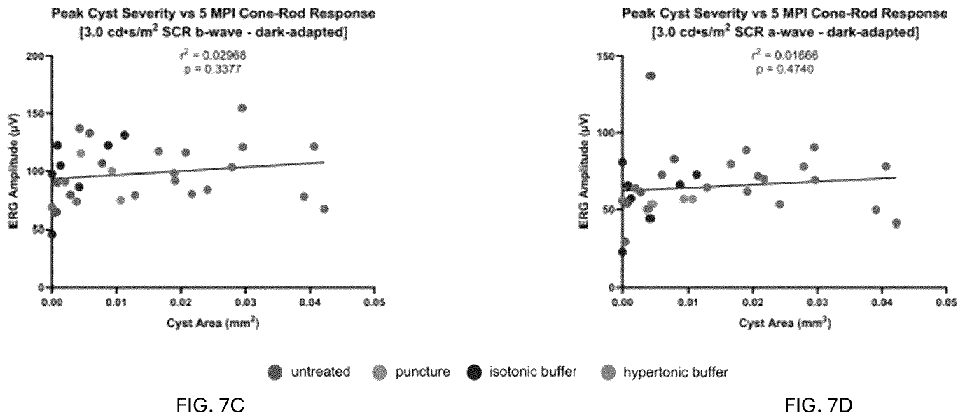

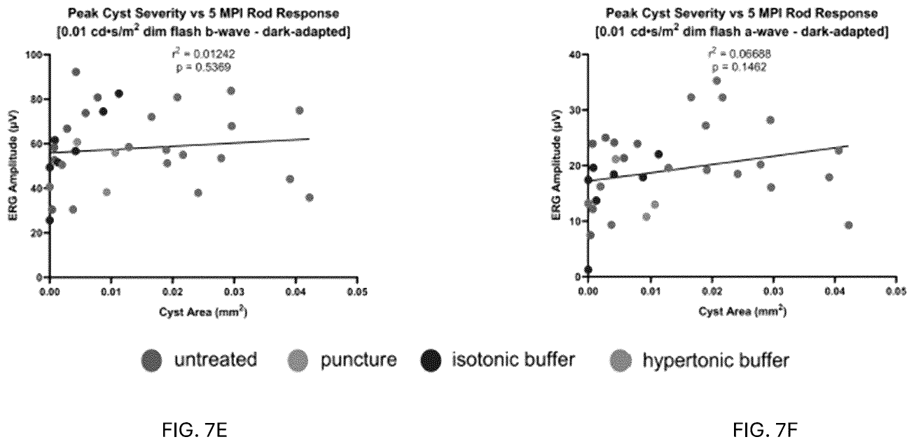

In light-adapted, cone-dominant, ERG metrics we found ERG amplitudes are negatively correlated with peak cyst severity in both the 5 Hz flicker and 3.0 flash (5 Hz flicker: r²=0.2027, p=0.0086; FIG. 7A) (3.0 flash b-wave: r²=0.1373, p=0.0338; FIG. 7B). These findings suggest that having a greater cyst area at any time during the disease course of a mouse is strongly associated with impaired signaling in cone-dominant electrical pathways long-term.

On dark-adapted, rod-dominant ERG metrics, there is no correlation between peak cyst area in either the a-wave or b-wave metrics (3.0 SCR b-wave: r²=0.02968, p=0.3377; FIG. 7C) (3.0 SCR a-wave: r²=0.01666, p=0.4740; FIG. 7D) (0.01 dim flash b-wave: r²=0.01242, p=0.5369; FIG. 7E) (0.01 dim flash a-wave: r²=0.06688, p=0.1462; FIG. 7F). Cyst severity, or the reduction thereof, does not appear to be associated with long-term rod signaling.

The negative correlation observed between cone-dominant ERG amplitudes and cyst severity indicates that having had severe cysts at any point in time is correlated with possessing lower cone-dependent ERG amplitudes over time. However, this correlation analysis cannot distinguish whether this is due to a decreased number of cones or possessing lower cone function. To determine whether there is a difference in the number of cone photoreceptor cells, we used an immunofluorescence assay to quantify cone density in eyes injected with hypertonic buffer compared to untreated eyes.

### 9. Increased Cone Density at 6 to 7-Months Old Suggests Cone Preservation Following Injection of Buffer Solution.

ONL thickness, a typical measure of photoreceptor cell survival, was not significantly different between eyes that received the sham surgical puncture, buffers with different tonicity, and untreated eyes (FIG. 5B). In the setting of a low overall percentage of cones in the mouse retina (2.8% of total photoreceptors) (26), the impact of the injections on cone survival may not be accurately determined by measuring ONL thickness on conventional OCTs.

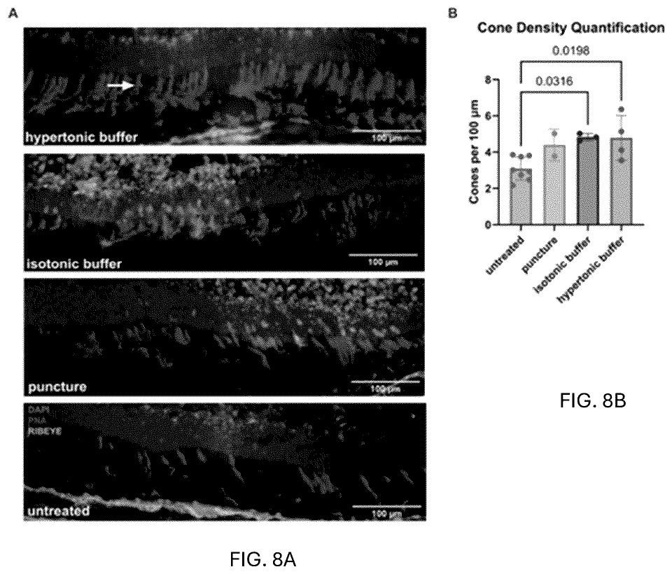

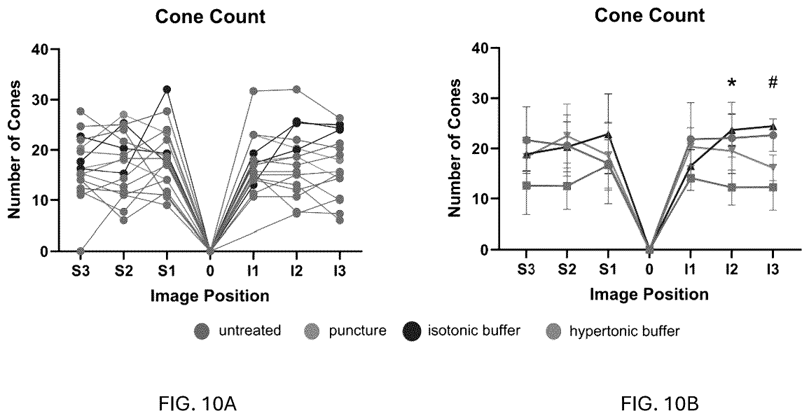

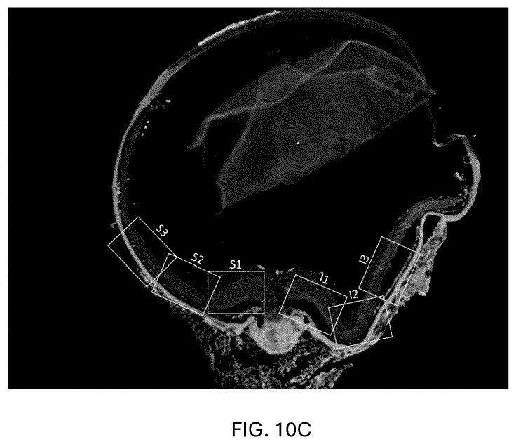

Immunohistochemistry was performed to measure cone cell survival. Retinal cryosections were stained with biotinylated-PNA, a marker for cone outer segments and pedicles. The number of PNA-positive cone outer segments were quantified for 6 images per eye and the number of cones per 100 μm of the retina was computed. Untreated Rs1-KO eyes had a mean of 3.09 cones per 100 μm, hypertonic buffer-injected eyes had a mean of 4.79 cones per 100 μm, isotonic buffer-injected eyes had a mean of 4.83 cones per 100 μm, and sham punctured eyes had a mean of 4.39 cones per 100 μm. Both hypertonic buffer-injected, and isotonic buffer-injected eyes had significantly higher cone density than untreated Rs1-KO eyes, suggesting improved cone survival compared to untreated eyes (hypertonic buffer vs. untreated: p=0.0198; isotonic buffer vs. untreated: p=0.0316; FIG. 8B). While both buffer-injected eyes had significantly more cones than untreated eyes, only hypertonic buffer-injected eyes showed significant improvements in light adapted ERG metrics. This suggests some mechanism beyond cone preservation could be responsible for the substantial improvements in ERG metrics seen in hypertonic buffer-injected eyes. Cone counts in specific regions of the retina inferior or superior to the optic nerve can be appreciated in FIGS. 10A-10C.

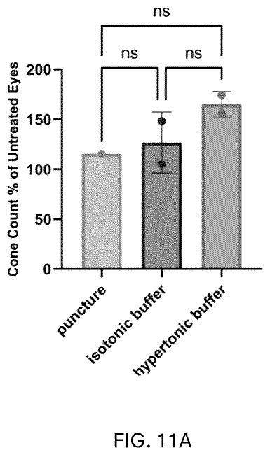

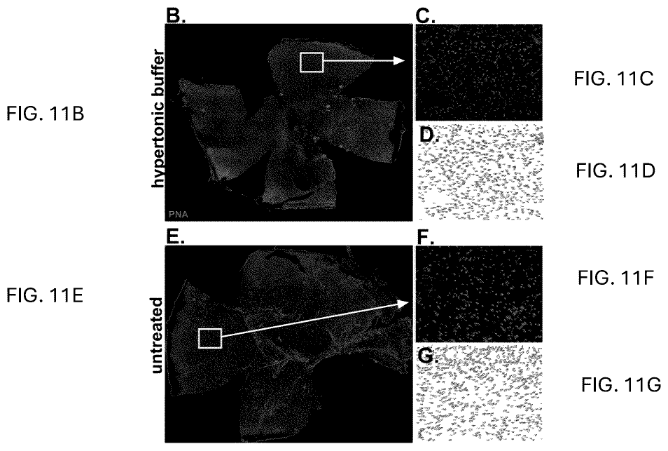

At 9 to 10 MPI, flat mounts of buffer injected, sham punctured, and untreated eyes were collected to determine if cone preservation persisted to later time points. The number of cones in two separate quadrants of an area approximately 350 μm², were quantified using ImageJ software, and the averaged cone density for that eye is reported. Cone density in the injected or sham punctured eye was normalized to the cone density in the untreated fellow eye and reported as a percent. Results of this experiment show a similar trend as manual cone counts derived at 6-7 months old, demonstrating that subretinal injections of buffer not only attenuate cysts in the short term but also lead to greater cone survival over time in the Rs1-KO mouse model. (FIGS. 11A-11G).

Together, this data shows that the subretinal injection of a buffer solution partially ameliorated intra-retinal cysts and leads to improved amplitudes in cone-specific ERGs as well as in functional light-adapted vision. The beneficial effect of the buffer solution after subretinal administration was partially mediated by the osmolarity of the buffer solution, with hypertonic buffer-injected eyes showing a more pronounced effect across all metrics.

### Discussion

Human trials of XLRS gene therapy are presently underway, working to overcome challenges associated with ocular immune reactions following intravitreal injection in past studies (11, 12). Our study initially aimed to explore the potential of a low-titer gene therapy in circumventing such immune reactions.

Initially, a subretinal injection of a 2×10⁹ vg/mL dose of AAV2/4-EF1α-RS1 in Rs1-KO mice did improve functional metrics such as cyst severity and cone electrical functioning, when compared to untreated Rs1-KO controls. However, it was surprising to observe that eyes injected with a hypertonic buffer as a sham injection also demonstrated improvements across all metrics compared to untreated Rs1-KO eyes. Even more unexpectedly, these eyes outperformed low-dose vector-treated eyes in cone electrical signaling and cyst severity scoring metrics, persisting to 5 MPI.

Previous studies utilizing higher titers delivered subretinally and intravitreally with an AAV2/4 vector and CMV promoter (27), and with a tYF modified capsid AAV (28) have demonstrated robust rescue of the retinal phenotype suggesting that sufficient RS1 expression is necessary to achieve optimal efficacy. However, toxicity was observed in some mice when the optimal dose was exceeded (28), highlighting the importance of understanding dose-response relationships in gene therapy for treating RS1. Considering the similarities in performance between the vector-treated and hypertonic buffer-injected eyes in the current study, it can be inferred that the efficacy observed with this very low dose vector was primarily attributed to its diluent buffer. Dose escalation in gene therapy is often accompanied by undesirable immunogenicity, yet a reduction of dose often results in loss of efficacy or sub-therapeutic outcomes. Together, these studies elucidate the tradeoff between achieving therapeutic efficacy and toxicity risks. However, the unexpectedly robust effect of the hypertonic buffer alone was not anticipated. While reductions in cysts for short periods following subretinal injection of a balanced salt solution have been reported in this model previously, this phenomenon has not been thoroughly explored (23).

To elucidate the mechanism of buffer-induced phenotypic rescue, ERG and OCT experiments were repeated after the subretinal injection of an isotonic buffer instead of the hypertonic buffer. Additionally, a sham group was added involving a scleral puncture to mimic injection without the introduction of subretinal fluid. The creation of a puncture in the sclera during the subretinal injection procedure causes leakage of a small amount of intra-ocular fluid, thus potentially lowering the intraocular pressure. Therefore, the addition of the sham puncture group accounts for this surgical effect.

Interestingly, hypertonic buffer-injected eyes exhibited a reduced cyst burden at 3 WPI, compared to untreated Rs1-KO eyes. By 1 MPI, a critical time point in cyst development as evidenced by natural history data (FIG. 1B), eyes across all experimental groups hypertonic buffer, isotonic buffer, and sham puncture, had a reduction in cyst severity compared to untreated Rs1-KO eyes. However, only hypertonic buffer-injected eyes maintained a reduction in cyst severity over untreated Rs1-KO eyes to 2 MPI. At 2 MPI, isotonic and hypertonic buffer-injected eyes had a more significantly reduced cyst burden than the sham puncture group, suggesting the introduction of either buffer solution has a greater effect on reducing cyst burden, with a slightly greater effect observed in the hypertonic buffer-injected eyes. An overall decrease in cysts was evident at 5 MPI across all groups, consistent with disease progression.

On ERG, isotonic buffer and hypertonic buffer-injected eyes showed improvement in cone-specific electrical signaling. However, the improvements were more pronounced in eyes that received the hypertonic buffer, persisting till 5 MPI. The combined effect of ERG and OCT experiments suggests the tonicity of the injected solution appears to play a minor yet important role in reducing overall cyst severity and enhancing the function of retinal cells, specifically cones, post-injection. The phenotypic improvements observed after the eyes received a sham puncture were more transient in nature compared to buffer-injected eyes.

Paradoxically, the improvements in cone-specific ERGs persisted despite age-related cyst resolution, and hypertonic buffer-injected eyes continued to possess higher ERG amplitudes to the study endpoint at 5 MPI. Age-related cyst resolution in this model accompanies a progressive loss of the laminar structure as seen on OCT. The thicknesses of the ONLs were not different between the experimental groups of mice, so our understanding of these functional improvements seen in hypertonic buffer-injected eyes long after cysts have naturally resolved remains unclear. Since cones are reported to represent only 2.8 percent of photoreceptors in mouse retinas, the effect of the injection on cone cell survival would not be reflected by ONL thickness measurements (26). The low cone-to-rod ratio and absence of a cone-rich region in the mouse retina present a limitation to this study, making it challenging to use mouse models for direct comparisons to human retinal conditions.

Immunohistochemistry revealed significantly higher cone density in both hypertonic buffer-injected and isotonic buffer-injected eyes compared to untreated eyes at 6 to 7-months, which could explain the improvement in cone specific ERG amplitudes and improvements in functional vision in light adapted environments. This finding suggests a potential cone-preserving mechanism is associated with the subretinal introduction of buffer, most impressively from the subretinal injection of a hypertonic buffer. While this does not fully explain the results as there is not a significant difference between hypertonic- and isotonic buffer-injected eyes, it does suggest that cone preservation at least partially underlies the observed improvements. The additional improvements seen in hypertonic buffer-injected eyes could be due to improved synaptic function, but further experiments are necessary to elucidate the mechanism further. Eyes were also collected and stained at 9 to 10-months for cone quantification. While hypertonic and isotonic buffer-injected eyes continued to show a trend of higher cone counts compared to untreated eyes, the difference was no longer statistically significant (FIGS. 11A-11G).

The long-term beneficial effects on XLRS phenotypes, including cone signaling, functional vision, and cyst severity, after the subretinal injection of a hypertonic buffer has not been previously reported. Previous studies propose that RS1 might have additional functions outside of its structural function and play a role in governing fluid distribution across retinal layers, potentially by engaging with Na/K-ATPase channels via a binding mechanism (24, 25). In the absence of a functional protein, this pathway has the potential to become dysregulated, resulting in the formation of fluid filled retinal cysts and creating a spatial separation of bipolar and photoreceptor cells. Despite the incomplete understanding of the mechanism, this phenomenon could provide an explanation for the observed benefits of carbonic anhydrase inhibitors (CAIs) in mitigating cyst development and enhancing visual function among XLRS patients (2).

Systemic infusions of hyperosmolar fluid have been shown to reduce edema in other organs, like the brain, in the setting of acute cerebral injury (29). With this precedent, strategies leveraging fluid exchange to promote the resolution of cysts in edematous retinal conditions merit further exploration. Aquaporins (AQP) represent a family of water-transporting channels with a pivotal role in upholding water homeostasis across various organ systems. Previously identified in the inner nuclear layer of the retina, studies suggest they are involved in regulating retinal water balance (30). When AQP was knocked out in mouse models, electroretinograms (ERGs) demonstrated decreased b-wave amplitudes and delayed latency in a series of ERGs with increasing light intensity (30). These findings are parallel to our observations in the Rs1-KO mouse natural history and underscore the connection between the electrical function of the eye and the fluid homeostasis of the retina.

Currently, these data suggest that the injection of a buffer that is hypertonic to the physiological environment best alleviates intraretinal cysts, promotes the survival of cone photoreceptors, and preserves retinal function in a mouse model of XLRS. Furthermore, these data suggested that managing the severity of cysts can have a long-term impact on the health of the retina. Analyzing the relationship between the peak severity of cysts in each animal and their long-term functional outcomes on ERG revealed a negative correlation between cyst severity and light-adapted ERG metrics. This correlation was seen regardless of the treatment cohort (untreated, hypertonic buffer, isotonic buffer, or sham puncture), suggesting that the light-adapted metrics, and thus cone function and/or survival, are closely related to severity of cysts. In the setting of greater cyst severity, cone function is more attenuated than rod function. This also suggests that long term cone function can be predicted by the degree of cyst reduction achieved by treatment, specifically at early time points when cysts are expected to be most severe. These results suggest the successful management of cysts can impact the long-term health and function of the retina.

While we have not determined the exact cause of the more robust increase in cones than rods observed in our study, several studies have shown that differing physiological states, such as hypoglycemia or hypoxia, affect rods and cones differently (31). Additionally, studies have shown that under certain conditions, infiltrating microglia selectively target rod photoreceptors for phagocytosis (32). Cones may be more sensitive to synaptic disruptions due to schisis. In our study, it remains unclear whether the degree of cyst resolution, the timing of cyst resolution, or another unmeasured factor contributes to the observed increases. The known differing sensitivities of rod and cone photoreceptors suggests that the factors leading to cell death in these photoreceptors may differ and could explain the cone-specific improvements observed in our results.

The relationship between cyst reduction and enhanced cone survival has not been previously reported and could have significant clinical implications. Managing XLRS symptoms using topical agents such as brinzolamide has been found to reduce cysts and have beneficial effect on visual function (8). However, whether managing cysts in XLRS could have long-term benefits on cone specific function is not clear. Our study provides compelling evidence that the attenuation of cysts may promote the survival of cones in the retina, and lead to better electrical cone signaling and functional vision in the long-term.

This study has provided evidence that the management of cysts through an osmolarity-related mechanism could have beneficial effects and could guide control arms of future gene therapy interventions. Hyperosmolar solutions could potentially have a role in treatment of XLRS, either as diluent for gene therapy vectors or on their own. This observation underscores the necessity of including a control group receiving the diluent buffer when evaluating the efficacy of a gene therapy vector in JXLR, as the buffer has unexpected effects on RS1 phenotypes. The effect of the buffer is specific to the XLRS murine phenotype and not a generic effect seen in all mouse strains. Improvements in cone function and survival, cyst severity, and light-adapted visual function after hypertonic buffer-injection is a surprising finding and suggests tonicity is a mediator of this effect. It can be speculated that this effect is due to the shifting of fluid out of the retinal space, where cysts form, and into the subretinal space where the choroidal vasculature can reabsorb cystic fluid. Although the exact mechanism is unknown, this study also demonstrates that lower peak cyst severity at critical cyst development is associated with improved electrical function, improvements in light-adapted visual acuity, and greater preservation of cones. This effect is partially modulated by the tonicity of the buffer and is most prominent in eyes injected with a hypertonic buffer. These findings have the potential to open new doors in the development of therapies for patients with XLRS and could shine a light on how fluid balance influences cyst resolution and disease severity.

### REFERENCES

1. Strupaite R, Ambrozaityte L, Cimbalistiene L, Asoklis R, Utkus A. X-linked juvenile retinoschisis: phenotypic and genetic characterization. Int J Ophthalmol. 2018; 11(11):1875-8.
2. Molday R S, Kellner U, Weber B H. X-linked juvenile retinoschisis: clinical diagnosis, genetic analysis, and molecular mechanisms. Prog Retin Eye Res. 2012; 31(3):195-212.
3. Sergeev Y V, Vitale S, Sieving P A, Vincent A, Robson A G, Moore A T, et al. Molecular modeling indicates distinct classes of missense variants with mild and severe XLRS phenotypes. Hum Mol Genet. 2013; 22(23):4756-67.
4. Sieving P A, MacDonald I M, Hoang S. X-Linked Congenital Retinoschisis. In: Adam M P, Feldman J, Mirzaa G M, Pagon R A, Wallace S E, Bean L J H, et al., editors. GeneReviews®. Seattle (WA) 1993.
5. Kiraly P, Fischer M D, Charbel Issa P. X-linked Retinoschisis with Near-Normal Macular Structure and Function. Ophthalmol Retina. 2023; 7(10):847.
6. Wolfensberger T J, Dmitriev A V, Govardovskii V I. Inhibition of membrane-bound carbonic anhydrase decreases subretinal pH and volume. Doc Ophthalmol. 1999; 97(3-4):261-71.
7. Apushkin M A, Fishman G A. Use of dorzolamide for patients with X-linked retinoschisis. Retina. 2006; 26(7):741-5.
8. Scruggs B A, Chen C V, Pfeifer W, Wiley J S, Wang K, Drack A V. Efficacy of topical brinzolamide in children with retinal dystrophies. Ophthalmic Genet. 2019; 40(4):350-8.
9. Thobani A, Fishman G A. The use of carbonic anhydrase inhibitors in the retreatment of cystic macular lesions in retinitis pigmentosa and X-linked retinoschisis. Retina. 2011; 31(2):312-5.
10. Kiraly P, Seitz I P, Abdalla Elsayed M E A, Downes S M, Patel C K, Charbel Issa P, et al.

Morphological and functional parameters in X-linked retinoschisis patients-A multicentre retrospective cohort study. Front Med (Lausanne). 2023; 10:1331889.
11. Cukras C, Wiley H E, Jeffrey B G, Sen H N, Turriff A, Zeng Y, et al. Retinal AAV8-RS1 Gene Therapy for X-Linked Retinoschisis: Initial Findings from a Phase I/IIa Trial by Intravitreal Delivery. Mol Ther. 2018; 26(9):2282-94.
12. Pennesi M E, Yang P, Birch D G, Weng C Y, Moore A T, Iannaccone A, et al. Intravitreal Delivery of rAAV2tYF-CB-hRS1 Vector for Gene Augmentation Therapy in Patients with X-Linked Retinoschisis: 1-Year Clinical Results. Ophthalmol Retina. 2022; 6(12):1130-44.
13. Reichel F F, Peters T, Wilhelm B, Biel M, Ueffing M, Wissinger B, et al. Humoral Immune Response After Intravitreal But Not After Subretinal AAV8 in Primates and Patients. Invest Ophthalmol Vis Sci. 2018; 59(5):1910-5.
14. Kjellstrom S, Bush R A, Zeng Y, Takada Y, Sieving P A. Retinoschisin gene therapy and natural history in the Rs1h-KO mouse: long-term rescue from retinal degeneration. Invest Ophthalmol Vis Sci. 2007; 48(8):3837-45.
15. Wiley L A, Burnight E R, Kaalberg E E, Jiao C, Riker M J, Halder J A, et al. Assessment of Adeno-Associated Virus Serotype Tropism in Human Retinal Explants. Hum Gene Ther. 2018; 29(4):424-36.
16. Zeng Y, Takada Y, Kjellstrom S, Hiriyanna K, Tanikawa A, Wawrousek E, et al. RS-1 Gene Delivery to an Adult Rs1h Knockout Mouse Model Restores ERG b-Wave with Reversal of the Electronegative Waveform of X-Linked Retinoschisis. Invest Ophthalmol Vis Sci. 2004; 45(9):3279-85.
17. Robson A G, Frishman L J, Grigg J, Hamilton R, Jeffrey B G, Kondo M, et al. ISCEV Standard for full-field clinical electroretinography (2022 update). Doc Ophthalmol. 2022; 144(3):165-77.
18. McCulloch D L, Kondo M, Hamilton R, Lachapelle P, Messias A M V, Robson A G, Ueno S. ISCEV extended protocol for the stimulus-response series for light-adapted full-field ERG. Doc Ophthalmol. 2019; 138(3):205-15.
19. Bush R A, Zeng Y, Colosi P, Kjellstrom S, Hiriyanna S, Vijayasarathy C, et al. Preclinical Dose-Escalation Study of Intravitreal AAV-RS1 Gene Therapy in a Mouse Model of X-linked Retinoschisis: Dose-Dependent Expression and Improved Retinal Structure and Function. Hum Gene Ther. 2016; 27(5):376-89.
20. Hsu Y, Bhattarai S, Thompson J M, Mahoney A, Thomas J, Mayer S K, et al. Subretinal gene therapy delays vision loss in a Bardet-Biedl Syndrome type 10 mouse model. Mol Ther Nucleic Acids. 2023; 31:164-81.
21. Hassan S, Hsu Y, Mayer S K, Thomas J, Kothapalli A, Helms M, et al. A visually guided swim assay for mouse models of human retinal disease recapitulates the multi-luminance mobility test in humans. Saudi J Ophthalmol. 2023; 37(4):313-20.
22. Ferguson L R, Dominguez J M, 2nd, Balaiya S, Grover S, Chalam K V. Retinal Thickness Normative Data in Wild-Type Mice Using Customized Miniature SD-OCT. PLoS One. 2013; 8(6):e67265.
23. Luna G, Kjellstrom S, Verardo M R, Lewis G P, Byun J, Sieving P A, Fisher S K. The effects of transient retinal detachment on cavity size and glial and neural remodeling in a mouse model of X-linked retinoschisis. Invest Ophthalmol Vis Sci. 2009; 50(8):3977-84.
24. Molday L L, Wu W W, Molday R S. Retinoschisin (RS1), the protein encoded by the X-linked retinoschisis gene, is anchored to the surface of retinal photoreceptor and bipolar cells through its interactions with a Na/K ATPase-SARM1 complex. J Biol Chem. 2007; 282(45):32792-801.
25. Plossl K, Royer M, Bernklau S, Tavraz N N, Friedrich T, Wild J, et al. Retinoschisin is linked to retinal Na/K-ATPase signaling and localization. Mol Biol Cell. 2017; 28(16):2178-89.
26. Jeon C J, Strettoi E, Masland R H. The major cell populations of the mouse retina. J Neurosci. 1998; 18(21):8936-46.
27. Scruggs B A, Bhattarai S, Helms M, Cherascu I, Salesevic A, Stalter E, et al. AAV2/4-RS1 gene therapy in the retinoschisin knockout mouse model of X-linked retinoschisis. PLoS One. 2022; 17(12):e0276298.
28. Hassan S, Hsu Y, Thompson J M, Kalmanek E, VandeLune J A, Stanley S, Drack A V. The dose-response relationship of subretinal gene therapy with rAAV2tYF-CB-hRS1 in a mouse model of X-linked retinoschisis. Front Med (Lausanne). 2024; 11:1304819.
29. Zhang H, Liu J, Liu Y, Su C, Fan G, Lu W, Feng L. Hypertonic saline improves brain edema resulting from traumatic brain injury by suppressing the NF-kappaB/IL-Ibeta signaling pathway and AQP4. Exp Ther Med. 2020; 20(5):71.
30. Li J, Patil R V, Verkman A S. Mildly abnormal retinal function in transgenic mice without Muller cell aquaporin-4 water channels. Invest Ophthalmol Vis Sci. 2002; 43(2):573-9.
31. Nihira M, Anderson K, Gorin F A, Burns M S. Primate rod and cone photoreceptors may differ in glucose accessibility. Invest Ophthalmol Vis Sci. 1995; 36(7):1259-70.
32. Zhao L, Zabel M K, Wang X, Ma W, Shah P, Fariss R N, et al. Microglial phagocytosis of living photoreceptors contributes to inherited retinal degeneration. EMBO Mol Med. 2015; 7(9):1179-97.

### Example 2 Intravitreal Hypertonic Buffer Injection Non-Invasive Therapeutic for XLRS

Here, whether intravitreal administration of a hypertonic buffer improves retinal function and structure in retinoschisin knockout (Rs1-KO) mice was investigated, and the efficacy of intravitreal administration of a hypertonic buffer was compared to subretinal delivery.

As detailed above, subretinal injections of a hypertonic phosphate-buffered saline (PBS), Gibco dPBS (−/−) (Catalog #10010) solution significantly improved cone function and reduced schisis cavities in Rs1-KO mice—a widely used model of XLRS. The hypertonic buffer appeared to modulate retinal fluid dynamics, potentially drawing excess fluid from schisis cavities into the subretinal space, where it could be absorbed by the choroidal vasculature. These findings provided early evidence that local osmotic modulation might influence disease severity in XLRS and offered a novel therapeutic avenue independent of gene replacement strategies.

Building on this finding, it was thought that intravitreal administration of the same hypertonic buffer would offer a similar therapeutic benefit to subretinal injections. If validated, this approach would be less invasive, more clinically feasible, and broadly applicable. To test this, a longitudinal study was conducted assessing cone and rod function, cystic area, and therapeutic durability following intravitreal hypertonic buffer injection in Rs1-KO mice. These outcomes were compared to untreated and isotonic buffer-treated controls, as well as to animals receiving subretinal hypertonic buffer injections. This work aimed to establish whether retinal osmotic modulation via the intravitreal route can serve as a viable, non-invasive, non-genetic, and repeatable therapeutic strategy for XLRS.

### Materials and Methods

### 1. Study Design

Rs1-KO animals between postnatal day (P) 15-P31 received intravitreal injections of either hypertonic or isotonic buffer. In treated animals, one eye received an intravitreal injection, while the contralateral eye served as untreated control. Both male and female Rs1-KO mice were included in the study (females were double knockout of Rs1 on both X chromosomes. To assess treatment efficacy and specificity, comparisons were made to eyes injected with isotonic buffer and to completely untreated Rs1-KO mice. In a separate comparison, outcomes from these intravitreal injections were also evaluated against prior results obtained from subretinal hypertonic buffer injections in the same mouse model.

### 2. Animal Husbandry and Ethics Statement

All animal procedures were conducted in accordance with the Institutional Animal Care and Use Committee (IACUC) guidelines under protocol #1041421 approved by the University of Iowa. The Rs1-KO mouse model, which recapitulates the human XLRS phenotype, was generously provided by Paul Sieving, M. D., Ph.D. The generation of the Rs1-KO line was previously described (Zeng et al., 2004).

### 3. Genotyping

Genotyping of Rs1-KO mice was carried out as previously described in Example 1 to confirm the presence of the Rs1 gene disruption (4).

### 4. Intravitreal Injection

Mice received a 1 μL intravitreal injection of either hypertonic or isotonic buffer (see, Example 1; (3)) into one eye under anesthesia. The injection was delivered into the vitreous space using a sharp 33-gauge, 0.5-inch (Hamilton 7803-15) needle under an operating microscope. The contralateral eye served as an untreated control in each animal.

### 5. Electroretinography and Optical Coherence Tomography

ERG and OCT imaging were performed to assess retinal function and structural integrity. Testing protocols and instrumentation were carried out as previously described in Example 1 (4). Retinal function was assessed by electroretinography (ERG), and cystic area was evaluated using optical coherence tomography (OCT) at 2 weeks post injection (WPI), and 2-, 3-, and 5-months post-injection (MPI) at the same time of day (morning). ERG measurements included modified ISCEV protocol (5) with scotopic and photopic responses, while OCT was used to measure schisis cavity area and quantification was performed using photoshop. Outcomes were compared across treated, sham, and untreated eyes, and with historical subretinal injection data.

### 6. Statistical Analysis

Statistical comparisons between groups and timepoints were performed using GraphPad Prism version 10.1. ANOVA and post hoc tests were used where appropriate. A p-value of <0.05 was considered statistically significant.

### Results

1. Cone Function Rescue after Intravitreal Hypertonic Buffer Injection

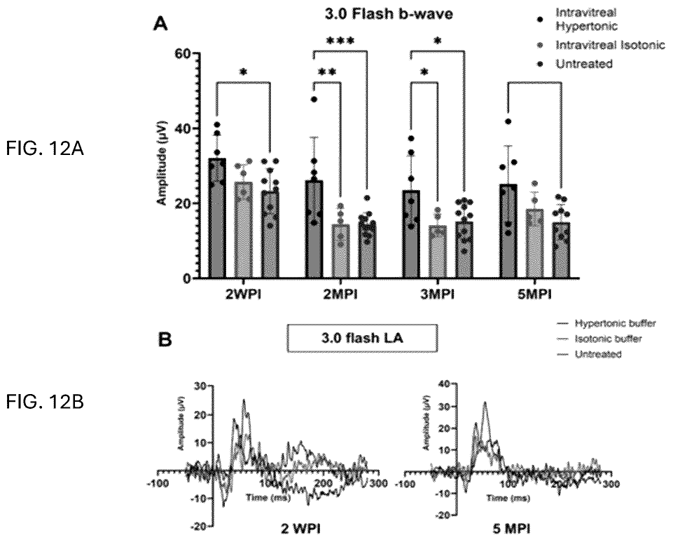

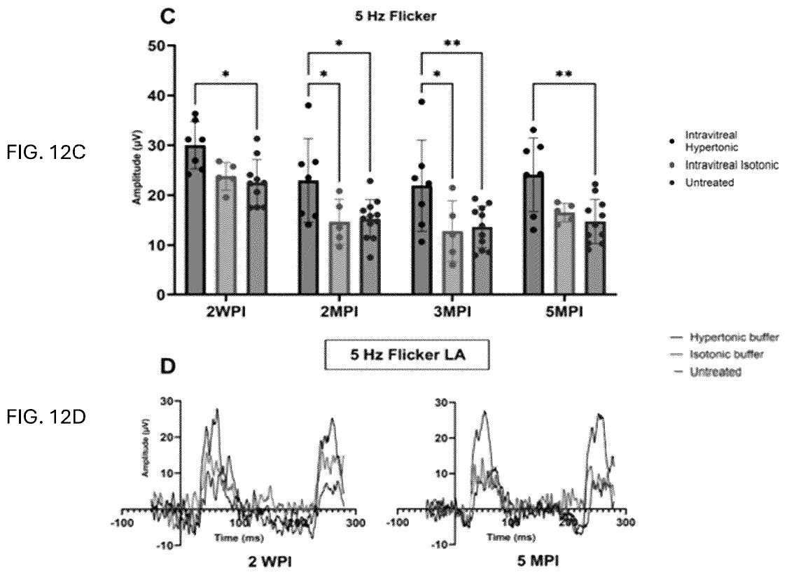

Following intravitreal injection of a hypertonic buffer into Rs1-KO mice, cone-mediated ERG responses-measured by the 3.0 flash and 5 Hz flicker light-adapted tests-showed significant improvement compared to untreated eyes. These enhancements were consistently observed at 2 weeks post-injection (WPI) (3.0 flash LA: P=0.012 (FIGS. 12A-12B); 5 Hz flicker: P=0.015 (FIGS. 12C-12D)), and sustained through 2-, 3-, and 5-months post-injection (MPI) (At 5MPI: 3.0 flash LA: P=0.0045 (FIGS. 12A-12B); 5 Hz flicker: P=0.0019 (FIGS. 12C-12D)) compared to the untreated eyes. The amplitudes of the b-wave of the 3.0 flash and 5 Hz flicker of the hypertonic treated group were also significantly higher than the isotonic treated group at 2 and 3 MPI but not at 5 MPI (FIGS. 12A-12D) suggesting a durable therapeutic benefit. This functional rescue highlights the potential of hypertonic buffer to mitigate cone dysfunction in XLRS through a non-gene therapy, non-invasive route.

### 2. Rod Function not Significantly Affected

Unlike cone responses, rod function assessed by dark-adapted 0.01 and 3.0 SCR ERG tests did not show statistically significant differences between hypertonic-treated and untreated groups. This suggests that the therapeutic effect of the hypertonic buffer is selective for cone cells.

### 3. Trend Toward Cyst Reduction Over Time

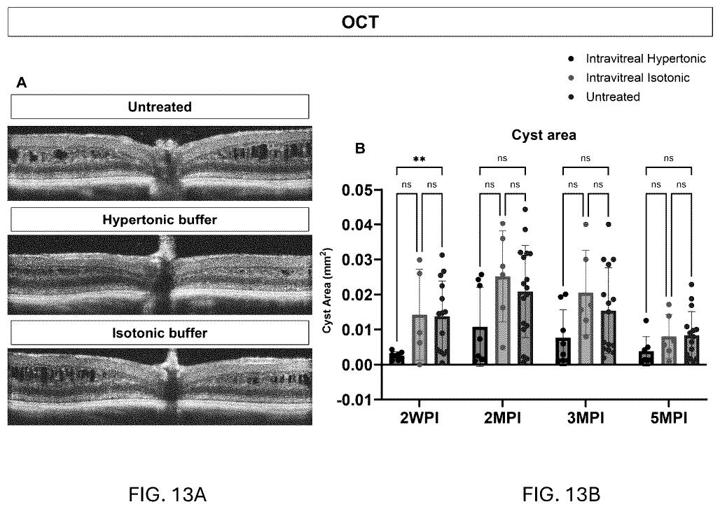

OCT imaging (FIG. 13A) revealed a significant reduction in schisis (cysts) area in hypertonic buffer-treated eyes compared to untreated controls at 2 WPI (P=0.002) (FIG. 12B). However, the rest of the timepoints and through 5 MPI, there was a trend observed in the reduction of cyst area (5MPI: P=0.16) (FIG. 13B). Although these reductions did not reach statistical significance, the consistent directionality supports the hypothesis that hypertonic solutions may facilitate fluid reabsorption and reduce cyst burden by altering osmotic gradients.

### 4. Comparable Efficacy Between Intravitreal and Subretinal Delivery

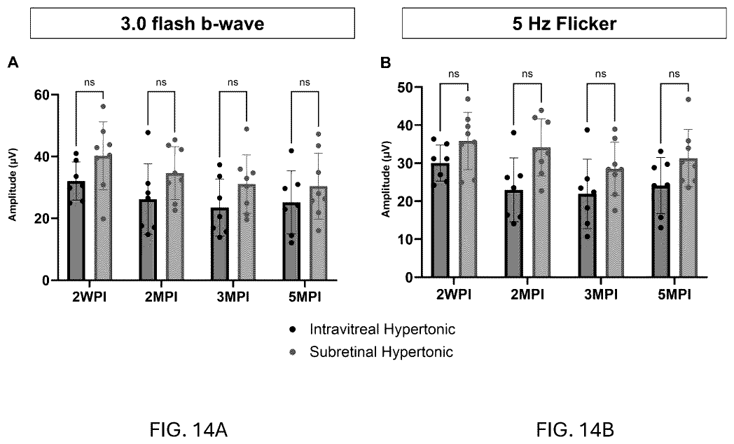

When comparing intravitreal and subretinal administration of the hypertonic buffer, both delivery methods yielded similar improvements in cone ERG responses. There was no significant difference between the two routes in either the 3.0 flash or 5 Hz flicker tests (FIGS. 14A-14B) across all timepoints. This finding is critical because intravitreal injections are less invasive and clinically more practical, supporting their potential as a feasible intervention in human XLRS patients.

### DISCUSSION

The results of this study demonstrate that intravitreal injection of hypertonic buffer significantly improves cone-mediated retinal function and reduces cystic changes in the Rs1-KO mouse model. These findings align with and extend previous work showing similar outcomes with subretinal delivery of hypertonic buffer (2), supporting the hypothesis that retinal fluid balance is a key contributor to disease severity and functional impairment in XLRS.

Specifically, intravitreal hypertonic buffer injections led to significantly increased b-wave amplitudes in light-adapted ERG tests (3.0 flash and 5 Hz flicker) at multiple timepoints compared to both untreated and isotonic-treated eyes. Although rod responses showed no significant improvement, the sustained enhancement in cone function indicates a selective and meaningful therapeutic impact on photopic pathways. One possible explanation for the more robust rescue of cone function compared to rods in our study builds on our previous work, where we proposed that cones may be more responsive to structural restoration due to their sensitivity to synaptic disruptions from schisis. It is also unclear whether the timing or extent of cyst resolution contributes to this differential rescue (2). Another explanation, drawn from broader retinal degeneration literature, is that cones often die secondarily due to loss of rod-derived support (6), giving them a longer survival window and making them more responsive to therapy. In contrast, rods undergo earlier, more complex degeneration that may be harder to reverse (6). However, in XLRS, whether rods degenerate before cones remains unknown.

The observed trend toward reduced cyst area in OCT imaging further supports a structural correlation to the functional recovery. While statistical significance in cyst reduction was most evident at the earliest timepoint (2WPI), the continued downward trend suggests that hypertonic buffer may promote gradual reabsorption of intraretinal fluid, potentially through osmotic gradient-driven movement toward the subretinal space and choroidal vasculature.

Importantly, the comparison between intravitreal and subretinal delivery routes revealed no statistically significant differences in ERG outcomes, suggesting that the less invasive intravitreal approach may offer comparable therapeutic benefit without the added risks associated with subretinal injections. This could represent a paradigm shift in the management of XLRS by favoring minimally invasive, non-gene therapy interventions.

Together, these results highlight osmotic modulation as a novel treatment strategy in XLRS and underscore the broader therapeutic potential of targeting fluid homeostasis in retinal diseases characterized by cystic degeneration. Further investigation is warranted to optimize dosing, duration of effect, and potential application to other inherited or acquired retinopathies with fluid accumulation phenotypes.

### REFERENCES

1—Molday R S, Kellner U, Weber B H. X-linked juvenile retinoschisis: clinical diagnosis, genetic analysis, and molecular mechanisms. Prog Retin Eye Res. (2012) 31:195-212. doi: 10.1016/j.preteyeres.2011.12.002
2—van der Veen I, Heredero Berzal A, Koster C, Ten Asbroek ALMA, Bergen A A, Boon C J F. The Road towards Gene Therapy for X-Linked Juvenile Retinoschisis: A Systematic Review of Preclinical Gene Therapy in Cell-Based and Rodent Models of XLRS. Int J Mol Sci. 2024 Jan. 19; 25(2):1267. doi: 10.3390/ijms25021267. PMID: 38279267; PMCID: PMC10816913.
3—Gehrke E J, Thompson J, Kalmanek E, Stanley S T, Laird J, Bhattarai S, Lobeck B, Mayer S, Mahoney A, Hassan S, Hsu Y, Drack A. An osmolarity dependent mechanism partially ameliorates retinal cysts and rescues cone function in a mouse model of X-linked retinoschisis. Front Med (Lausanne). 2024 Oct. 15; 11:1302119. doi: 10.3389/fmed.2024.1302119. PMID: 39473494; PMCID: PMC11518720.
4—Hassan S, Hsu Y, Thompson J M, Kalmanek E, VandeLune J A, Stanley S, Drack A V. The dose-response relationship of subretinal gene therapy with rAAV2tYF-CB-hRS1 in a mouse model of X-linked retinoschisis. Front Med (Lausanne). 2024 Feb. 13; 11:1304819. doi: 10.3389/fmed.2024.1304819. PMID: 38414621; PMCID: PMC10898246.
5—Robson, A. G., Frishman, L. J., Grigg, J., Hamilton, R., Jeffrey, B. G., Kondo, M., Li, S., & McCulloch, D. L. (2022). ISCEV Standard for full-field clinical electroretinography (2022 update). Documenta ophthalmologica. Advances in ophthalmology, 144(3), 165-177. https://doi.org/10.1007/s10633-022-09872-0
6—Petit L, Punzo C. Gene therapy approaches for the treatment of retinal disorders. Discov Med. 2016 October; 22(121):221-229. PMID: 27875674; PMCID: PMC5142441.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method of preventing or treating X-linked retinoschisis (XLRS) in a subject in need thereof, the method comprising: administering an aqueous composition to an intravitreal space of an eye of a subject in need thereof.

2. The method of claim 1, wherein the aqueous composition comprises a buffered saline solution, wherein the buffered saline solution is a hypertonic buffered saline solution or an isotonic buffered saline solution.

3. The method of claim 2, wherein the buffered saline solution comprises about 100 mM to about 400 mM NaCl.

4. The method of claim 2, wherein the buffered saline solution comprises about 180 mM NaCl or about 155 mM NaCl.

5. The method of claim 1, wherein the subject in need thereof has one or more RS1 gene mutations.

6. The method of claim 1, wherein the subject in need thereof has one or more intraretinal cysts.

7. The method of claim 1, wherein the aqueous composition does not include a therapeutic agent.

8. The method of claim 7, wherein the therapeutic agent is a gene therapy.

9. The method of claim 3, wherein the buffered saline solution comprises about 100 mM to about 400 mM NaCl, PBS, and 0.001 wt. % of a polyoxyethylene-polyoxypropylene block copolymer.

10. The method of claim 1, further comprising topically administering one or more carbonic anhydrase inhibitors to the eye of the subject and/or reducing a pressure within a vitreous cavity of the eye of the subject.

11. A method of reducing intraretinal cysts and/or preventing the formation of intraretinal cysts in a subject in need thereof, the method comprising administering an aqueous composition to an intravitreal space of an eye of a subject in need thereof.

12. The method of claim 11, wherein the aqueous composition comprises a buffered saline solution, wherein the buffered saline solution is a hypertonic buffered saline solution or an isotonic buffered saline solution.

13. The method of claim 12, wherein the buffered saline solution comprises about 100 mM to about 400 mM NaCl.

14. The method of claim 12, wherein the buffered saline solution comprises about 180 mM NaCl or about 155 mM NaCl.

15. The method of claim 11, wherein the subject in need thereof has one or more RS1 gene mutations and/or one or more intraretinal cysts.

16. A method of treating X-linked retinoschisis (XLRS) and/or reducing intraretinal cysts in a subject in need thereof, the method comprising:
administering a topical and/or a subconjunctival hypertonic solution to the eye of the subject;
rapid lowering of intraocular pressure in the eye of the subject using medical and/or surgical techniques; and
administering an aqueous composition to an intravitreal space of the eye of the subject.

17. The method of claim 16, wherein the aqueous composition comprises a buffered saline solution, wherein the buffered saline solution is a hypertonic buffered saline solution or an isotonic buffered saline solution.

18. The method of claim 17, wherein the buffered saline solution comprises about 100 mM to about 400 mM NaCl.

19. The method of claim 17, wherein the buffered saline solution comprises about 180 mM NaCl or about 155 mM NaCl.

20. The method of claim 16, wherein the topical and/or a subconjunctival hypertonic solution comprises one or more carbonic anhydrase inhibitors.


SEQUENCE LISTING (3 sequences)

SEQ ID NO: 1 - DNA, 21 bp, synthetic construct
  source 1..21: mol_type = other DNA
taggggcccacatcttccaac

SEQ ID NO: 2 - DNA, 24 bp, synthetic construct
  source 1..24: mol_type = other DNA
gttcttcggacgcctcgtcaacac

SEQ ID NO: 3 - DNA, 27 bp, synthetic construct
  source 1..27: mol_type = other DNA
gtgacaaagagccacacaacagtgacc